(19) United States
(12) Patent Application Publication
(10) Pub. No.: US 2024/0043818 A1
(43) Pub. Date: Feb. 8, 2024

(54) Engineered Cells for Increased Collagen Production

(71) Applicant: Northeastern University, Boston, MA (US)

(72) Inventors: Tim John MASTOVICH, Boston, MA (US); Michael FITZGERALD, Santa Clara, CA (US); Jessie Lyn GIFFORD, Boston, MA (US); Andrew C. HOROWITZ, Mansfield, MA (US); Megan Jayne POWELL, Malden, MA (US); Jeffrey William RUBERTI, Lexington, MA (US)

(21) Appl. No.: 18/031,486

(22) PCT Filed: Oct. 15, 2021
(86) PCT No.: PCT/US2021/055315

§ 371 Date: Apr. 12, 2023

Related U.S. Application Data
(60) Provisional application No. 63/092,433, filed on Oct. 15, 2020.

(51) Int. Cl. C12N 9/22; C12N 15/11; C12P 21/02; A61K 38/39; A61K 8/65; A61Q 19/00; A61L 27/24; A61L 26/00

(52) U.S. Cl. CPC C12N 9/22; C12N 15/11; C12P 21/02; A61K 38/39; A61K 8/65; A61Q 19/00; A61L 27/24; A61L 26/0033; C12N 2310/20; C12N 2800/80; A61K 2800/10; A61L 2430/16; A61L 2430/02

(57) ABSTRACT

Cells for increased collagen production are engineered by a novel CRISPR cellular engineering process. The process can be carried out using human cells or even patient-harvested cells. Collagen produced by the cells has a low risk of immunogenicity when implanted into human patients compared to collagen produced by non-human cells. Cell culture media including chemical additives are also provided to have a further positive effect on collagen production.

### CROSS-REFERENCE TO RELATED APPLICATIONS

This application claims priority to U.S. Provisional Application No. 63/092,433, filed 15 Oct. 2020, which is incorporated by reference herein in its entirety.

### STATEMENT REGARDING FEDERALLY SPONSORED RESEARCH OR DEVELOPMENT

This invention was made with government support under Grant No. EY029167 awarded by the National Institutes of Health. The government has certain rights in the invention.

### BACKGROUND

On average there are 33 million musculoskeletal injuries in the United States per year, and fifty percent of these injuries involve tendons and ligaments (e.g., rotator cuff, anterior cruciate ligament) and are due to trauma or degeneration (Wu, et al., 2017). Tendons and ligaments are collagen-based soft tissues and are often subject to insufficient healing after injury. Further, the average reported recovery time for these tendon and ligament injuries is 12 weeks, and injured tendons may never fully return to the preinjury state (nhs.uk/conditions/hand-tendon-repair/recovery/). To speed up this lengthy recovery time, treatments are being developed involving implantation of type I collagen patches following surgical repair of these injured soft tissues. The surgical patches can help to bridge the gap between ends of torn tendons and can avoid stretching the injured tissue to keep it strong and to prevent reinjury (orthocarolina.com/media/how-does-a-patch-repair-a-rotator-cuff-tear). Even after these surgical interventions, the patient often does not regain the strength or mobility of the preinjury state. Currently the type I collagen for these patches is derived from animals and, consequently, such treatments present a significant risk of immune system rejection when implanted into human patients (Vig, et al., 2019). One example is the Zimmer© collagen repair patch, which is made for reinforcement of rotator cuff repairs. The patch is composed of an acellular collagen derived from porcine dermis (Yao, et al., 2005). The patch has shown promise over other biomaterial scaffolds, but its mechanical properties are not strong enough for tendon or ligament repair, and the manufacturing process used to sterilize and purify the patch compromises the collagen structure (OKeefe, et al., 2013). There is a need for improved sources of collagen for tissue repair and reconstruction in human patients.

### SUMMARY

The present technology provides engineered cells, including human cells, capable of greatly enhanced collagen production and methods of using them to obtain collagen for treatment of medical conditions without the risk of undesired immune reactions. Using the present methodology, cells can be obtained from a patient in need of collagen supplementation for treatment of a medical condition and then engineered to produce large amounts of the patient's own collagen for implantation, including auto-telocollagen, auto-procollagen, or auto-atelocollagen. Additionally, the method can be used with same species (e.g., human donor cells) to produce allo-telocollagen, allo-procollagen, or allo-atelocollagen.

The cellular engineering process overcomes bottlenecks in the collagen synthesis pathway via genetic engineering using CRISPR, together with optional use of chemical additives in the cell growth media that stimulate translation and post-transcriptional modifications involved in collagen synthesis. Genetic modifications can be made to human cells such as corneal fibroblasts using CRISPR activation (CRISPRa) to increase the expression of one or more genes responsible for the transcription, translation, and post-translational processing of collagen, including type I collagen. The collagen can be used, for example, in collagen patches for soft tissue repairs, and can be produced for a fraction of the current market cost for human collagen. Further, collagen provided by the present methods lacks telopeptide damage because there is no need for pepsin extraction. Because the collagen can be from a human source or from cells derived from the patient to be treated with the collagen, fewer screening and purification procedures are needed.

One aspect of the technology is an engineered cell capable of enhanced collagen biosynthesis. The cell has been engineered to perform CRISPR-based activation (CRISPRa) of a targeted gene related to collagen biosynthesis by the cell. As a result of transfection or transduction with components of the CRISPRa system, the cell expresses an endonuclease deficient Cas9 (dCas9) protein fused to a transcriptional activator protein (dCas9-activator) and also expresses a guide RNA (gRNA) specific for the targeted gene. The engineered cell is capable of increased collagen biosynthesis compared to a non-engineered cell of the same type. The collagen biosynthesis rate of the cell has been increased, such as, for example, by at least 10-fold, at least 20-fold, at least 30-fold, at least 40-fold, at least 50-fold, at least 60-fold, at least 70-fold, at least 80-fold, at least 90-fold, at least 100-fold, at least 200-fold, or at least 300-fold as a result of the CRISPRa engineering. The cell is preferably of a type that, as naturally occurring, produces the desired collagen type whose biosynthesis is activated by CRISPRa in the engineered cell. For example, the cell can be of a type selected from the group consisting of fibroblasts of any desired tissue or organ, chondrocytes, osteoblasts, epithelial cells, endothelial cells, mesenchymal cells, pericytes, hematopoietic cells, and fibrocytes.

In another aspect of the technology, the engineered cell described above has been engineered using CRISPRa to increase the expression of one or more genes selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1. Preferably, the cell has been engineered to increase the expression of 2 or more, 3 or more, 4 or more, or 5 or more of those genes in the same cell. For example, the cell can express gRNA molecules including the nucleotide sequence of any one or more of SEQ ID NOS:1-156.

In yet another aspect of the technology, the engineered cell has been engineered to perform CRISPRa of one or more further targeted genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin. The increased expression of these genes contributes to the post-translational processing or secretion of collagen from the engineered cell into the medium.

In still another aspect of the technology, the engineered cell has been engineered by CRISPRa to increase the expression of one or more collagen genes and one or more TGF β genes. Preferably, the cell also has been engineered to increase the expression of one or more propeptidase genes. Each of the activated genes can have its expression increased by at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 200-fold, or at least 300-fold. Each separate activated gene can be activated to a different degree, or to a similar degree, compared to other activated genes in the cell.

Regarding the aspect of the technology described above, the cell preferably can be engineered to increase expression of one or more collagen genes selected from the group consisting of COL1A1, COL1A2, COL2A1, COL3A1, COL4A1, COL4A2, COL4A3, COL4A4, COL4A5, COL4A6, COL5A1, COL5A2, and COL5A3. It also preferably can be activated to increase expression of one or more TGF-βgenes selected from the group consisting of TGF-β1, TGF-β2, and TGF-β3. The cell also preferably can be engineered to increase expression of one or more propeptidase genes selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

The engineered cell preferably contains or produces mRNA capable of expressing a deactivated or “dead” Cas9 protein, lacking endonuclease activity, which is fused to one or more transcription activator proteins. For example, the transcription activators can be selected from the group consisting of VP64, p65, Rta (e.g., encoded by Epstein-Barr virus BRLF1), VPR (a combination of VP64, p65, and Rta), MS2, HSF1, and SAM (a combination of MS2, p65, and HSF-1). The activator SunTag also can be used to activate a gene and provide a fluorescent signal for transcriptional activation (Tannenbaum et al. (2014)).

Yet another aspect of the present technology is a cell culture containing any of the above described engineered cells. The cell culture can contain only a single cell type which are all identical, or it can contain a mixture of two or more differently engineered cell types, either derived from the same cell or type of cell, or derived from different types of cells. The culture can be derived from one or more cells obtained from a subject in need of collagen supplementation, thus providing a pathway to producing sufficient amounts of collagen that is genetically, biochemically, and immunologically identical to collagen of the subject's tissues. Preferably the cell culture is capable of at least 5, at least 10, at least 20, or at least 30 passages without substantial loss of collagen biosynthesis rate, or has been immortalized.

Still another aspect of the technology is a method for engineering a cell to provide enhanced collagen biosynthesis. The method includes the steps of: (a) providing a collagen-producing cell, a first nucleic acid molecule encoding a dCas9-activator, and a second nucleic acid molecule specific for a target gene related to collagen biosynthesis; and (b) transfecting or transducing the cell with said first and second nucleic acid molecules. The cell becomes capable of expressing said dCas9-activator and said gRNA, and the target gene is activated, whereby collagen synthesis by the cell is increased.

Yet another aspect of the present technology is a method of producing collagen. The method includes the steps of: providing the cell culture described above; growing the cell culture in a bioreactor under conditions in which collagen is biosynthesized by the cells of the cell culture; and harvesting and purifying collagen from the bioreactor. Preferably, the growth of the cells in the bioreactor is in the presence of a modulator or stimulator of collagen biosynthesis, such as an agent selected from acetaldehyde, ascorbate, hyaluronic acid, p-aminopropionitrile, transforming growth factor beta (TGF-β), insulin-like growth factor 1 (IGF-1), glutamine, and combinations thereof. Preferably, the cell growth medium of the bioreactor is concentrated after collagen biosynthesis by eliminating water and solutes having a molecular weight less than 50 Daltons while retaining higher molecular weight compounds, whereby propeptide cleavage of the collagen is accelerated.

Even another aspect of the technology is a kit of parts for engineering a cell to enhance biosynthesis of collagen by the cell. The kit includes: (i) a first nucleic acid molecule encoding a dCas9-activator protein; (ii) one or more second nucleic acid molecules specific for one or more target genes related to collagen biosynthesis; and (iii) optionally one or more reagents for transfecting or transducing a cell with the first and second nucleic acid molecules.

Another aspect of the technology is a medical device that contains an engineered cell, or cell culture, as described above, or collagen produced by such a cell or cell culture. The device can be an ex vivo device capable of producing collagen by engineered cells derived from a clinical subject's own cells. Alternatively, the device can be implantable in the subject's body, and can either contain engineered cells derived from cells of the subject, or can contain collagen produced by such cells; the collagen can be attached to a surface of the medical device, or can be contained in a reservoir for delivery into a tissue of the subject. The medical device can also serve as a collagen delivery device, whereby the device is disposed outside the subject's body, or is worn by the subject, and delivers collagen into a tissue of the subject's body.

Still another aspect of the technology is a method of treating a mammalian subject, such as a human, having a medical condition characterized by insufficiency of collagen. The method includes: obtaining collagen produced by a cell culture as described above, wherein the engineered cells of the culture are derived from the subject or from one or more other subjects of the same species; and administering the collagen to the subject. The collagen can be administered by injection into a tissue or placement within the body during surgery. The collagen can be in the form of a membrane, sheet, pad, solution, or gel, or contained within a cell scaffold, bandage, or wound dressing, or can be in the form of a coating on an implanted medical device. The medical condition can be, for example, a wound, a torn ligament or tendon, a bone fracture, damaged cartilage, an eye condition, a condition requiring cosmetic treatment or surgery, a dermatological condition, skin wrinkles or scars, or a burn.

The present technology can be further summarized by the following list of features.
1. An engineered cell capable of enhanced collagen biosynthesis, wherein the cell has been engineered to perform CRISPR-based activation (CRISPRa) of a targeted gene related to collagen biosynthesis by the cell, wherein the cell expresses an endonuclease deficient Cas9 (dCas9) protein fused to a transcriptional activator protein (dCas9-activator) and a guide RNA (gRNA) specific for the targeted gene, wherein the engineered cell is capable of at least 10-fold, at least 20-fold, at least 30-fold, at least 40-fold, at least 50-fold, at least 60-fold, at least 70-fold, at least 80-fold, at least 90-fold, at least 100-fold, at least 200-fold, or at least 300-fold higher collagen biosynthesis compared to a non-engineered cell of the same type.
2. The engineered cell of feature 1, wherein the targeted gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.
3. The engineered cell of feature 2, wherein the gRNA expressed by the cell and specific for said targeted gene comprises the nucleotide sequence of any of SEQ ID NOS:1-156.
4. The engineered cell of any of the preceding features, wherein the cell has been engineered to perform CRISPRa of one or more further targeted genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.
5. The engineered cell of any of the preceding features that expresses gRNAs specific for two or more of said targeted genes, and each of the two or more targeted genes is activated.
6. The engineered cell of feature 5, wherein one or more collagen genes and one or more TGF β genes are targeted;
   wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3;
   wherein the one or more TGF-βgenes are selected from the group consisting of TGF-β1, TGF-β2, and TGF-β3; and
   wherein the cell expresses gRNAs specific for said one or more collagen genes and said one or more TGF-βgenes.
7. The engineered cell of feature 6, wherein the collagen genes are selected from COL1A1 and COL1A2 and the TGF-βgenes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:13-16, and the TGF-β3 gRNA comprises the nucleotide sequence of any of SEQ ID NOS:9-12.
8. The engineered cell of feature 6 or feature 7, wherein one or more propeptidase genes are further targeted, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.
9. The engineered cell of feature 8, wherein the propeptidase genes ADAMTS2 and BMP-1 are targeted, and wherein the ADAMTS2 gRNA comprises the nucleotide sequence of any of SEQ ID NOS:69-72 and the BMP-1 gRNA comprises the nucleotide sequence of any of SEQ ID NOS:153-156.
10. The engineered cell of any of the preceding features, wherein said transcriptional activator is selected from the group consisting of VP64, p65, Rta, VPR (a combination of VP64, p65, and Rta), MS2, HSF1, SAM (a combination of MS2, p65, and HSF-1), and SunTag.
11. The engineered cell of feature 10, wherein the dCas9-activator is dCas9-VPR.
12. The engineered cell of any of features 1-11, wherein the cell has been transfected to express said dCas9-activator and said gRNA or gRNAs.
13. The engineered cell of any of features 1-11, wherein the cell has been transduced to express said dCas9-activator and said gRNA or gRNAs.
14. The engineered cell of any of the preceding features, wherein the cell is derived from a cell type selected from the group consisting of fibroblasts, mesenchymal cells, myofibroblasts, osteoblasts, chondrocytes, and induced pluripotent stem cells.
15. The engineered cell of feature 14, wherein the cell is derived from a human corneal fibroblast.
16. The engineered cell of any of the preceding features, wherein the cell is derived from a cell obtained from a mammalian subject in need of collagen administration.
17. A cell culture comprising the engineered cell of any of the preceding features.
18. The cell culture of feature 17 that is immortalized.
19. A method for engineering a cell to provide enhanced collagen biosynthesis, the method comprising the steps of:
   (a) providing the cell, a first nucleic acid molecule encoding a dCas9-activator, and a second nucleic acid molecule specific for a target gene related to collagen biosynthesis; and
   (b) transfecting or transducing the cell with said first and second nucleic acid molecules;
      whereby the cell becomes capable of expressing said dCas9-activator and said gRNA, and the target gene is activated.
20. The method of feature 19, wherein the target gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1
21. The method of feature 20, wherein the gRNA comprises the nucleotide sequence of any of SEQ ID NO:1 to SEQ ID NO:156.
22. The method of any of features 19-21, wherein the cell has been engineered to perform CRISPRa of one or more further targeted genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.
23. The method of any of features 20-22, wherein the cell is transfected with two or more second nucleic acid molecules, each specific for a different target gene, whereby each of the target genes is activated.
24. The method of feature 23, wherein one or more collagen genes and one or more TGF β genes are targeted;
   wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3;
   wherein said TGF-β genes are selected from the group consisting of TGF-β1, TGF-β2, and TGF-β3; and
   wherein the cell expresses gRNAs specific for said one or more collagen genes and said one or more TGF-β genes.
25. The method of feature 24, wherein the collagen genes are selected from COL1A1 and COL1A2 and the TGF-β genes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:13-16, and the TGF-β3 gRNA comprises the nucleotide sequence of any of SEQ ID NOS:9-12.
26. The method of feature 24 or feature 25, wherein one or more propeptidase genes are further targeted, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.
27. The method of feature 26, wherein the propeptidase genes ADAMTS2 and BMP-1 are targeted, and wherein the ADAMTS2 gRNA comprises the nucleotide sequence of any of SEQ ID NOS:69-72 and the BMP-1 gRNA comprises the nucleotide sequence of any of SEQ ID NOS:153-156.
28. The method of any of features 19-27, wherein the dCas9-activator is dCas9-VPR.
29. The method of any of features 19-28, wherein the cell is derived from a cell type selected from the group consisting of fibroblasts, myoblasts, osteoblasts, chondrocytes, and induced pluripotent stem cells.
30. The method of feature 29, wherein the cell is derived from a human corneal fibroblast.
31. The method of any of features 19-30, wherein the cell is transduced using a lentiviral vector in step (b).
32. The method of any of features 19-31, wherein step (a) includes obtaining a sample from a mammalian subject in need of collagen administration, or from a different mammalian subject of the same species, and deriving the provided cell from the sample.
33. A kit for engineering a cell to enhance biosynthesis of collagen by the cell, the kit comprising:
   (i) a first nucleic acid molecule encoding a dCas9-activator protein;
   (ii) a second nucleic acid molecule comprising or encoding a crRNA specific for a target gene related to collagen biosynthesis; and
   (iii) optionally one or more reagents for transfecting or transducing a cell with the first and second nucleic acid molecules.
34. The kit of feature 33, wherein the target gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.
35. The kit of feature 34, wherein the crRNA comprises the nucleotide sequence of any of SEQ ID NOS:1-156.
36. The kit of feature 33, wherein two or more second nucleic acid molecules are provided, each comprising or encoding a crRNA specific for a different target gene.
37. The kit of feature 36, wherein the two or more second nucleic acid molecules comprise or encode crRNAs specific for one or more collagen genes and one or more TGF β genes;
   wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3; and
   wherein said TGF-β genes are selected from the group consisting of TGF-β1, TGF-β2, and TGF-β3.
38. The kit of feature 37, wherein the collagen genes are selected from COL1A1 and COL1A2 and the TGF-β genes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:13-16, and the TGF-β3 gRNA comprises the nucleotide sequence of any of SEQ ID NOS:9-12.
39. The kit of feature 37 or feature 38, wherein the two or more second nucleic acids further comprise or encode crRNAs specific for one or more propeptidase genes, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.
40. The kit of feature 39, wherein the two or more second nucleic acid molecules comprise or encode crRNAs specific for propeptidase genes ADAMTS2 and BMP-1, and wherein the ADAMTS2 gRNA comprises the nucleotide sequence of any of SEQ ID NOS:69-72 and the BMP-1 gRNA comprises the nucleotide sequence of any of SEQ ID NOS:153-156.
41. The kit of any of features 33-40, wherein the second nucleic acid molecules further comprise or encode crRNAs specific for one or more genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.
42. The kit of any of features 33-41, wherein the dCas9-activator is dCas9-VPR.
43. A medical device comprising the engineered cell of any of features 1-16.
44. The medical device of feature 43 that is implantable in a subject's body.
45. A method of producing collagen, the method comprising the steps of:
   (a) providing the cell culture of feature 17 or 18;
   (b) growing the cell culture in a bioreactor under conditions in which collagen is biosynthesized by the cells of the cell culture; and
   (c) harvesting and purifying collagen from the bioreactor.
46. The method of feature 45, wherein step (b) is performed in the presence of a modulator of collagen biosynthesis.
47. The method of feature 46, wherein the modulator is selected from the group consisting of acetaldehyde, ascorbate, hyaluronic acid, p-aminopropionitrile, transforming growth factor beta (TGF-β), insulin-like growth factor 1 (IGF-1), glutamine, and combinations thereof.
48. The method of feature 47, wherein the modulator is a combination of ascorbate and β-aminopropionitrile or a combination of ascorbate, acetaldehyde, and β-aminopropionitrile.
49. The method of feature 47, wherein the modulator is β-aminopropionitrile, and wherein crosslinking of collagen is reduced or prevented compared to absence of β-aminopropionitrile.
50. The method of any of features 45-49, wherein step (b) is performed in the presence of application of mechanical strain to the cells.
51. The method of feature 50, wherein mechanical strain is induced using cells adhered to a substrate, beads, or a scaffold.
52. The method of any of features 45-51, further comprising, between steps (b) and (c): (b1) concentrating the biosynthesized collagen in the cell growth medium, whereby propeptide cleavage of the biosynthesized collagen is enhanced.
53. The method of any of features 45-52, wherein the collagen produced is a type selected from the group consisting of collagen types I-V.
54. The method of feature 53, wherein the collagen is type I collagen.
55. A method of treating a mammalian subject having a medical condition characterized by insufficiency of collagen, the method comprising:
   (a) performing the method of feature 32 and thereby obtaining collagen produced by cells derived from the mammalian subject, or a different mammalian subject of the same species; and
   (b) administering the collagen to the subject.
56. The method of feature 55, wherein a medical device is used to administer the collagen.
57. The method of feature 55 or 56, wherein the medical device is selected from the group consisting of a burn/wound covering or dressing, an osteogenic and/or bone filling material, a device having an antithrombogenic surface, a device having a therapeutic enzyme immobilization surface, a collagen patch, a closure graft, an implant operative to provide collagen, a corneal implant, a bandage contact lens, a collagen-based membrane, and a collagen-based drug delivery device.
58. The method of any of features 55 to 57, wherein the medical condition is selected from the group consisting of a wound, a torn ligament or tendon, a bone fracture, damaged cartilage, an eye condition, a condition requiring cosmetic treatment or surgery, a dermatological condition, skin wrinkles or scars, and a burn.
59. A method of performing a cosmetic treatment to a human subject, the method comprising:
   (a) performing the method of feature 32, thereby obtaining collagen produced by cells derived from the mammalian subject or other subject of the same mammalian species; and
   (b) administering the collagen obtained in step (a) to the subject.

As used herein, the prefix “auto” refers to a product (e.g., auto-procollagen, auto-telocollagen, or auto-atelocollagen) derived from cells of the same subject as the subject undergoing treatment using the product.

As used herein, the prefix “allo” refers to a product (e.g., allo-procollagen, allo-telocollagen, or allo-atelocollagen) derived from cells of the same species, but not the same subject, as the subject undergoing treatment using the product.

As used herein, “procollagen” refers to a newly synthesized, inactive collagen subject to activation by cleavage of propeptides from the procollagen.

As used herein, “telocollagen” refers to an active form of collagen, capable of assembly to form collagen fibrils, that is created by cleavage of propeptides from procollagen.

As used herein, “atelocollagen” refers to collagen stripped of its telopeptides, such as by pepsin digestion.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1 is an illustration of a process of transducing cells for CRISPRa activation of collagen synthesis.

FIG. 2 depicts plasmids required to construct the lentiviral vector used for the transduction process shown in FIG. 1.

FIG. 3 illustrates a process of constructing the lentiviral vector used for the transduction process shown in FIG. 1.

FIG. 4 shows a collagen standard curve obtained for a SirCol™ Soluble Collagen Assay (biocolor.co.uk/product/sir).

FIG. 5 shows a plot of collagen production and cell count as a function of transfection reagent concentration.

FIG. 6 shows a plot of collagen production as a function of transfection reagent concentration and days in culture.

FIG. 7 shows a graph of transient collagen production rate over a six-day period. Error bars are shown for collagen production during days 5-6 (shorter culture times did not show a statistically significant difference).

FIG. 8 shows a plot of collagen production as a function of chemical additive. Bars annotated with an asterisk were below assay detection limit.

FIG. 9 shows a plot of collagen production as a function of chemical additive group. Bars annotated with an asterisk were below assay detection limit.

FIG. 10 shows a plot of cell viability as a function of chemical additive.

FIG. 11 shows a schematic representation of an apparatus for collagen production under conditions of fluid shear. Cells are seeded on stacked glass plates configured for fluid flow across the plates and optimization of surface area.

FIG. 12 shows a schematic representation of a bioreactor with floating glass beads serving as cell carriers in the medium.

### DETAILED DESCRIPTION

The present technology provides novel human cells engineered to increase collagen production. The cells are produced utilizing a CRISPR activation (CRISPRa) cellular engineering process to induce rapid collagen production from a variety of cell types, including human cells. Genetic modifications are introduced into cells that naturally produce a desired type of collagen using CRISPRa to increase the expression of one or more genes responsible for the transcription, translation, and/or post-translational processing of collagen. Collagen production from the engineered cells can be further stimulated by growing the engineered cells in the presence of one or more chemical additives in the cell culture medium to achieve even greater rates of collagen production. The synthesized collagen can be isolated, purified, and then used in collagen patches, gels, or other forms for soft tissue repairs.

The present inventors have achieved dramatically increased collagen production by applying cellular engineering to certain bottlenecks in collagen biosynthesis. Collagen has a unique protein structure. Collagen consists of three amino acid chains which form a triple helix. The primary amino acid sequence found in collagen is glycine-X-hydroxyproline or glycine-proline-X, where X is any other amino acid. The significant amount of glycine (every third amino acid) allows the helix to form in a tight configuration making the molecule structurally resistant to stress (Lodish, et al., 2000). Type I collagen molecules are 300 nm long and 1.5 nm in diameter. Each collagen molecule is composed of a characteristic right-handed triple helix which is composed of two alpha 1 chains and one alpha 2 chain. Each chain contains 1050 sequential amino acids. Once the individual collagen molecules are formed, they pack together side by side to form fibers with a diameter of about 10-300 nm and about a 67 nm gap between the “head” and “tail” of adjacent molecules (Schleip, 2012). N-terminal to C-terminal covalent bonds stabilize interactions of collagen molecules that are located adjacent to one another. The periodic pattern of molecule packing creates striations which are visible by electron microscopy. The links between molecules facilitate collagen packing stability to form strong fibrils. In addition to type I collagen, Table 1 lists examples of other collagen types and their features.

TABLE 1
Collagen Types and Key Features.

| Collagen | |
|---|---|
| Type | Features |
| I | Most abundant collagen in human body: found in tendons, ligaments, skin, |
| | artery walls, cornea, endomysium, fibrocartilage, intestines, bones, uterus, and |
| | teeth. |
| II | Hyaline cartilage; Found in vitreous humor of the eye, tendons, liver, and |
| | cartilage. |
| III | Reticular fibers; Found in artery walls, skin, intestines, liver, scar tissue, and |
| | uterus. |
| IV | Functions in kidney filtration system; Found in basal lamina, the epithelium |
| | secreted layer of the basement membrane, placenta, and amniotic membrane. |
| V | Interstitial tissue; Found in cornea, ligaments, and intestines. |
| VI | Interstitial tissue; Found in ligaments, vasculature, skin, and cartilage. |
| VII | Forms anchoring fibrils; Found in epidermal/dermal junctions. |
| VIII | Component of some endothelial cells. |
| IX | FACIT collagen. |
| X | Mineralizing and hypertrophic cartilage. |
| XI | Cartilage. |
| XII | FACIT collagen. |
| XIII | MACIT collagen. |
| XIV | FACIT collagen (undulin). |
| XVI | FACIT collagen. |
| XVII | Transmembrane collagen. |
| XVIII | Source of endostatin. |
| XIX | FACIT collagen. |
| XX | FACIT collagen. |
| XXI | FACIT collagen. |
| XXIII | MACIT collagen. |
| XXV | MACIT collagen. |
| XXIX | Epidermal collagen. |

Collagen synthesis occurs primarily in fibroblasts through a process that spans both the intracellular and extracellular space. Type I collagen production is primarily controlled by two genes: Collagen type I alpha 1 (COL1A1), a strip of 17,533 base pairs on chromosome 17 that occurs after the 50,184,096th base pair, and Collagen type I alpha 2 (COL1A2), a strip of 36,671 base pairs on chromosome 7 that occurs after the 94,394,561st base pair (NIH, 2019).

Most genes responsible for collagen production contain an exon-intron pattern with an average number of exons ranging from 3 to 117. Depending on the type of cell and collagen, there are multiple transcription initiation sites and exon splicing mechanisms which result in different mRNA species (Gelse, et al., 2003). Specifically for type I collagen production, the pro-alpha 1 and pro-alpha 2 chain genes are transcribed from the COL1A1 and COL1A2 genes, respectively. During this phase of collagen production, the pre-mRNA undergoes both splicing and capping. The cellular transcription activity depends on cell type and is regulated by numerous growth factors and cytokines. Some of these growth factors include members of the Transforming Growth Factor Beta (TGF-β) family, fibroblast-growth factors, and insulin-like growth factors (Gelse, et al., 2003). The efficacy of these growth factors depends on the cell type.

Once translation has occurred, the collagen is in a pre-pro-polypeptide chain phase, and it moves to the lumen of the RER for post translational modifications (Wu & Crane, 2019; Lodish, et al., 2000). These molecules intrude into the lumen by the assistance of receptors that recognize the signal recognition domain of the collagen molecules (Gelse, et al., 2003). Three major modifications are made to convert this chain to procollagen. The first modification is the removal of the signal peptide on the N-terminal of the peptide chain by the enzyme signal peptidase. Efficient cleavage by the signal peptidase requires smaller amino acids (i.e., alanine, glycine, serine) just before the cleavage site, so that the signal peptidase 1 (SPase 1) can properly cleave the terminal (Tuteja, 2005).

The second modification is the hydroxylation, or addition of hydroxyl groups (—OH), of lysine and proline residues by hydroxylase enzymes (FIG. 5). Specifically this reaction is catalyzed by prolyl 3-hydroxylase, prolyl 4-hydroxylase, and lysyl hydroxylase (Gelse, et al., 2003). This modification requires several cofactors including ascorbate, ferrous ions, 2-oxoglutarate, and oxygen. The extent of hydroxylation is both species- and temperature-dependent. The hydroxylation modification of pre-pro-collagen is essential to the formation of intramolecular hydrogen bonds and, therefore, collagen thermal stability and monomer and collagen fibril integrity.

The third modification is the glycosylation of hydroxylysine with glucose and galactose. During this modification, glucosyl and galactosyl residues are placed on the hydroxyl groups of hydroxylysine. Hydroxylation of specific proline and lysine residues (non-hydroxylated) in the middle of the chain are catalyzed by hydroxylysyl galactosyltransferase and galactosylhydroxylysylglucosyltransferase enzymes bound to the endoplasmic reticulum membrane. Oligosaccharides are also bound to asparagine residues in the C-terminal propeptide of procollagen (Lodish, et al., 2000).

After these three post-translational modifications are made, the glycosylated and hydroxylated chains assemble into a triple helix by folding, much like a zipper, as intrachain disulfide bonds are “zipped” together. The helix consists of two alpha 1 (I) chains and one alpha 2 (I) chain subunits. This assembly consists of three left-handed helices configured in a 1050 amino acid long right-handed coil, which forms from the C-terminus to N-terminus in the endoplasmic reticulum before further post-translational changes take place. C-propeptides also play a role in the assembly of the peptide chains into a collagen monomer (Gelse, et al., 2003).

After processing and procollagen assembly, the triple-helical molecule moves to the Golgi apparatus for final modifications and packaging inside the tubular portion of the complex known as vesicular tubular clusters (Wu & Crane, 2019; Bonfanti, et al., 1998). Within these clusters, the procollagen aggregates and is packaged within the Golgi compartment into secretory vesicles and released for transportation to the extracellular space.

Outside of the cell, collagen peptidase enzymes cleave the unraveled propeptides on the N-terminal and C-terminal to remove the ends of the molecule and convert the molecule to tropocollagen. The protease that performs the propeptide cleavage is procollagen C-proteinase. The tropocollagen terminates on both ends with telopeptides, which can be an issue in regard to antigenicity and immunogenicity (Stuart, et al., 1982; Lynn, et al., 2004). Collagen molecules have telopeptides on either side of their chains. The telopeptides do not form the typical triple helical formation and contain the amino acid hydroxylysine. Hydroxylysine residues form crosslinks at the C-terminal of one molecule and the N-terminal of two adjacent molecules (collplant.com/technology; Lodish, et al., 2000). These telopeptides also can be a source of immunogenicity if the collagen is transplanted into another species, or even intraspecies (Stuart, et al., 1982; Lynn, et al., 2004; Uchio, et al., 2000). The triple helical region of collagen is conserved across species. Although variations in amino acid sequences within the helix differ by less than a few percent between species, up to fifty percent of the amino acid sequence in the telopeptides can differ between species (Lynn, et al., 2004). Due to this high interspecies variation in this region of the molecule, telopeptides are thought to be the primary contributing factor to immune responses post collagen implantation.

The final extracellular step is fibrillogenesis. Fibril-forming collagen molecules spontaneously self-assemble into ordered fibrillar structures. Long thin collagen fibrils are formed when lysyl oxidase covalently bonds lysine and hydroxylysine molecules. This behavior is encoded in the collagen structure. Fibril orientation depends on the type of tissue (Gelse, et al., 2003). Each fibril has a diameter of about 100 nm after the molecules are packed together side by side, although fibril diameter can range from 25-500 nm.

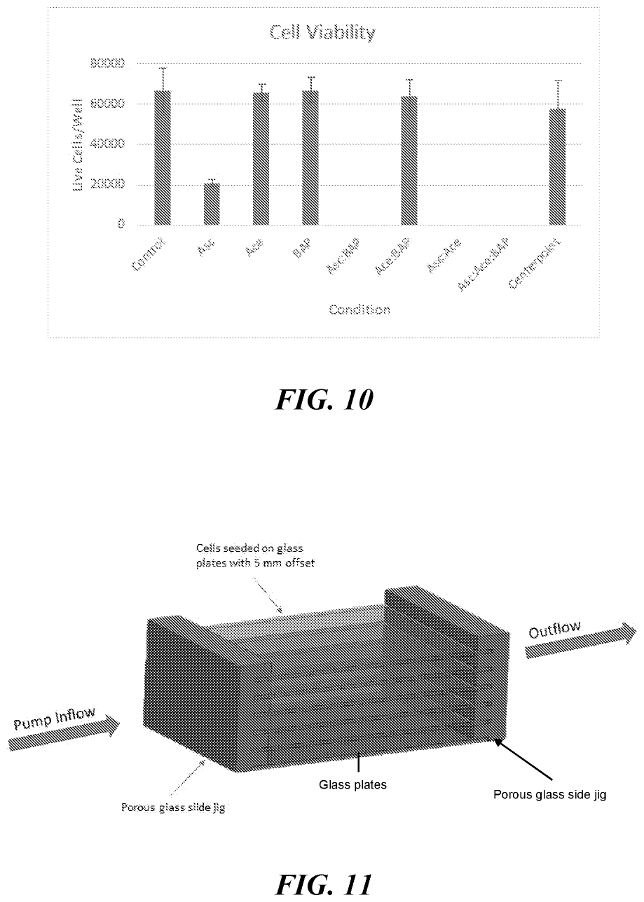

Clustered regularly interspaced short palindromic repeats (CRISPR) and CRISPR associated protein-coding genes (Cas) are a group of proteins used by the immune system of prokaryotes. CRISPR associated protein 9 (Cas9) can cut DNA, and is a highly efficient DNA targeting enzyme that has been modified for gene editing research applications. The CRISPR-Cas9 system is made up of four main parts: the Cas9 enzyme, guide RNA (gRNA), protospacer adjacent motif (PAM) sequence, and matching host DNA (matching genomic sequence). Cas9 is an endonuclease enzyme that utilizes an approximately 20 base pair section of guiding RNA to recognize, unzip, and induce double-strand breaks in DNA (Biolabs, 2019). Guide RNA (gRNA) directs the CRISPR-Cas9 system where to go in the genome and can result in the process of CRISPR-Cas9 cutting the host DNA, and then letting natural DNA repair processes incorporate an inserted gene of interest into the host's genome at a very particular point in the host genome (as defined by the gRNA). The gRNA has two main components, CRISPR RNA (crRNA) and trans-activating crRNA (tracrRNA) (FIG. 10A, Prior Art, Packer, 2016). The crRNA section contains an 18-23 base pair sequence that directs the Cas9 protein to the desired complementary genome location for editing. This crRNA is bound via hybridization to a section of tracrRNA that matures and helps direct the crRNA. A linker loop can be attached to these two sections to form a single guide RNA (sgRNA) (Packer, 2016).

The matching host DNA is a 18-23 base pair sequence that is in or near the promoter region of the gene of interest. This sequence must be immediately followed by an NGG (representing any base pair followed by two guanines) or ‘PAM sequence’. In order to determine what base pairs should be targeted by the gRNA in a given gene, computational biology tools have been developed to find PAM sequences and base pair sequences of interest (CRISPR Guide Design Software, Pelligrini, 2016). These base pairs are ranked for how well the Cas9 system is able to bind to that sequence without accidentally attaching to other similar sequences in the genome. Additionally, manufacturers, like Dharmacon™, have proprietary software that is used to compute highly specific binding sites for gRNA.

The present inventors elected to use CRISPR gene activation (CRISPRa) to engineer cells that produce much greater amounts of collagen than naturally occurring cells. In CRISPRa, a deactivated or dead Cas9 enzyme (dCas9) without endonuclease activity is used together with a guide RNA (gRNA or sgRNA) to locate to a specific gene target. The dCas9 can be fused to one or more transcriptional activator proteins. The resulting fusion protein is referred to herein as “dCas9-activator”. The one or more activators fused to dCas9 can be, for example, VP64 (a tetramer of the Herpes Simplex Viral Protein 16) or VPR (VP64 bound to β53 and an R transcription factor)). For example, the VPR activator can be dCas9 from S. pyrogenes fused to VP64-β65-Rta. Other activators or combinations of activators can be selected according to cell type or gene to be activated. The dCas9 does not cut the bound DNA, but acts to upregulate expression of the targeted gene. The activator domain fused to the dCas9 causes transcriptional activation by recruiting transcription complexes to the promoter regions of these genes.

An important design consideration when performing CRISPRa is selecting where dCas9 binds to the gene; generally, a position on the gene's promoter is selected. While specificity of a gRNA sequence and locations of its PAM sequences can be predicted using computer algorithms, the location on the promoter and its resulting effectiveness is variable. While the promoter area for effectiveness of CRISPRa is generally 50-400 base pairs upstream of the transcription start site, the most effective location for activation varies between genes, and some locations are completely ineffective (Mohr, et al., 2016).

There are two practical ways to deliver the CRISPR-Cas9 system for performing CRISPRa (i.e., the dCas9-activator and gRNA for genes to be activated) into a cell: transfection and transduction.

Transfection is the delivery of nucleic acids (typically the mRNA corresponding to the transcribed gene) into a cell and subsequent translation of the mRNA by the host cell. When performing CRISPRa using transfection, the CRISPR-Cas9 system is usually expressed for about 24-48 hours. Transfection of cells with the CRISPRa components can be performed by microinjection, electroporation, or use of ribonucleoprotein (RNP) complexes to deliver the mRNAs. Transient expression using transfection is simpler and less expensive than transduction, and decreases the odds of off-target activation due to its short expression window. Further, the use of mammalian expression vectors allows for transfection that is less transient than traditional, non-mammalian transfection vectors.

Commercial kits are available for performing CRISPRa by transfection of cells. For example, Dharmacon (horizondiscovery.com) offers a protocol and reagents for pooled transfection of gRNA and dCas9 mRNA for culture in a 96-well plate using the DharmaFECT Duo Transfection Reagent. The protocol can be scaled up to a 48-well plate or further in order to harvest more collagen, such as for more accurate collagen quantification while engineering cells for increased collagen production. Additionally, when two or more different sets of gRNA are used to activate two or more genes simultaneously, appropriate adjustments can be made to the amount of dCas9 mRNA and the amount of transfection reagent. For example, in order to simultaneously activate expression of the COLA1, COL!A2, and TGF-β3 genes in a human cell, the materials can include CRISPRa Human COL1A1 crRNA pool, CRISPRa Human COL1A2 crRNA pool, CRISPRa Human TGF-β3 crRNA pool, CRISPR-Cas9 Synthetic tracrRNA, Edit-R GFP dCas9-VPR mRNA, DharmaFECT Duo Transfection Reagent, 10 mM Tris-HCl Buffer pH 7.4, and serum-free medium. Example reagents needed for a pooled transfection of human corneal fibroblasts for increased collagen production are shown below in Table 2. Only one pooled crRNA purchase is necessary as target crRNAs can be mixed and matched in one pool purchase (one pool contains four times the minimum single crRNA needed for CRISPRa), but having more than the minimum crRNA necessary can lead to better gene activation (CRISPR Guide Design Software).

TABLE 2
Reagents Needed for Pooled Transfection

| | | Amount needed for one |
|---|---|---|
| | Catalog | CRISPRa experiment (48 well |
| Item Name | Number | plate) |
| CRISPRa Human | P-010502- | .54 nmol (electroporation) |
| COL1A1 crRNA pool | 010005 | .0025 nmol (transfection reagent) |
| CRISPRa Human | P-004758- | .54 nmol (electroporation) |
| COL1A2 crRNA pool | 010005 | .0025 nmol (transfection reagent) |
| CRISPRa Human TGF- | P-012562- | .54 nmol (electroporation) |
| β1 crRNA pool | 010005 | .0025 nmol (transfection reagent) |
| CRISPR-Cas9 | U-002005-05 | .54 nmol (electroporation) |
| Synthetic tracrRNA | | .0025 nmol (transfection reagent) |
| dCas9-VPR mRNA | CAS12024 | 5 μg (electroporation) |
| (also in GFP and | | .2 μg (transfection reagent) |
| Puromycin variants) | | |
| DharmaFECT 1 | T-2001-01 | .8 μL |
| Transfection Reagent | | |
| 10 mM Tris-HCl Buffer | B-006000-100 | .05 mL |
| pH 7.4 | | |

The CRISPRa Human COL1A1 crRNA pool includes a pool of individual RNA sequences complimentary to different regions of the COL1A1 promoter (see target sequences SEQ ID NOS:1-4 in Table 3).

TABLE 3
CRISPR Target Sequences for Promoters of Targeted Genes Related
to Collagen Biosynthesis

| | DNA Target Sequence | Target Gene |
|---|---|---|
| SEQ ID NO: 1 | GCTGCGAAGAGGGGAGATGT | COL1A1 (human) |
| SEQ ID NO: 2 | GGGGAGGCAGAGCTGCGAAG | COL1A1 (human) |
| SEQ ID NO: 3 | CCGGCCCCCAATTTGGGAGT | COL1A1 (human) |
| SEQ ID NO: 4 | GGAACCCTGCCCCTCGGAGA | COL1A1 (human) |
| SEQ ID NO: 5 | AAGGGCCTCCACCAATGGGA | COL1A2 (human) |
| SEQ ID NO: 6 | CGCAGAGGAGGGAGCGAATG | COL1A2 (human) |
| SEQ ID NO: 7 | GGGGAAGGGACGTGGCCACG | COL1A2 (human) |
| SEQ ID NO: 8 | GGGAGGGCGGGAGGATGCGG | COL1A2 (human) |
| SEQ ID NO: 9 | TTAACATCGTGCAGCAAAAG | TGF-ß3 (human) |
| SEQ ID NO: 10 | GAGGGCGCGGGACCCGGTAG | TGF-ß3 (human) |
| SEQ ID NO: 11 | GCAAAAGAGGCTGCGTGCGC | TGF-ß3 (human) |
| SEQ ID NO: 12 | CCGGGACCGGGGGACCAGGA | TGF-ß3 (human) |
| SEQ ID NO: 13 | GCGGCCAAGCGCCACCAAAG | TGF-ß1 (human) |
| SEQ ID NO: 14 | GAGCCCGCCCACGCGAGATG | TGF-ß1 (human) |
| SEQ ID NO: 15 | CCCCGCGGGCGGCTCAGAGC | TGF-ß1 (human) |
| SEQ ID NO: 16 | CCGCCCACGCGAGATGAGGA | TGF-ß1 (human) |
| SEQ ID NO: 17 | GAGCTCTCCCCGAACCGTTG | TGF-ß2 (human) |
| SEQ ID NO: 18 | ATGAGGACCGCTGTGGGTAA | TGF-ß2 (human) |
| SEQ ID NO: 19 | GTGGAAATGAGGACCGCTGT | TGF-ß2 (human) |
| SEQ ID NO: 20 | CTCGTGGTCTAAGTAACGAG | TGF-ß2 (human) |
| SEQ ID NO: 21 | GCCGCAGCCCCGGGTTTGGG | COL2A1 (human) |
| SEQ ID NO: 22 | GCCACTCGGCGCACTAGGGG | COL2A1 (human) |
| SEQ ID NO: 23 | CAGGCCACTCGGCGCACTAG | COL2A1 (human) |
| SEQ ID NO: 24 | CCAAGCCGGACCCCCCTCTC | COL2A1 (human) |
| SEQ ID NO: 25 | CGGCTCTCATATTTCAGAAA | COL3A1 (human) |
| SEQ ID NO: 26 | GCAGTTGTTAACTTCATAAG | COL3A1 (human) |
| SEQ ID NO: 27 | TGTGGGTTGTGTCTTCTATA | COL3A1 (human) |
| SEQ ID NO: 28 | TAACTTCTAGGACCCAGGGT | COL3A1 (human) |
| SEQ ID NO: 29 | GGGCGCGAGGGGTTGGGACG | COL4A1 (human) |
| SEQ ID NO: 30 | GACCCTGCGGCGCGGGTAAG | COL4A1 (human) |
| SEQ ID NO: 31 | GAGCGCGCGGCCCGGGAGTG | COL4A1 (human) |
| SEQ ID NO: 32 | GCGCACTGCAGCCACACTCC | COL4A1 (human) |
| SEQ ID NO: 33 | TGGAGCCGCCGCACCCGGGA | COL4A2 (human) |
| SEQ ID NO: 34 | CCGGGTGCGGCGGCTCCAAG | COL4A2 (human) |
| SEQ ID NO: 35 | GCGGACAGCTAGCTCTCGGA | COL4A2 (human) |
| SEQ ID NO: 36 | GCCCCATGGTGGCGCGCCCG | COL4A2 (human) |
| SEQ ID NO: 37 | CGTGCCCAGGAGGCGAGAAA | COL4A3 (human) |
| SEQ ID NO: 38 | TCTACCCGGGCATCGTGCCC | COL4A3 (human) |
| SEQ ID NO: 39 | GGCACGATGCCCGGGTAGAA | COL4A3 (human) |
| SEQ ID NO: 40 | AGGGACACTGCCTGGTAAGT | COL4A3 (human) |
| SEQ ID NO: 41 | CGTGCCCAGGAGGCGAGAAA | COL4A4 (human) |
| SEQ ID NO: 42 | TCTACCCGGGCATCGTGCCC | COL4A4 (human) |
| SEQ ID NO: 43 | GGGAAGTGGGGTGCGGTCGG | COL4A4 (human) |
| SEQ ID NO: 44 | GGATCCAGGGTAAGGGGTTA | COL4A4 (human) |
| SEQ ID NO: 45 | GAGTGACGCTCAGTTATTTG | COL4A5 (human) |
| SEQ ID NO: 46 | GACTGCACCGGCAACCTGCG | COL4A5 (human) |
| SEQ ID NO: 47 | GGTACGCACACCAATGAGAT | COL4A5 (human) |
| SEQ ID NO: 48 | GTCACTCCCTCGCAGGTTGC | COL4A5 (human) |
| SEQ ID NO: 49 | TTAGCGTATAGGTCTCTAAG | COL4A6 TSS* P1 (human) |
| SEQ ID NO: 50 | CGGGCCCATCTGTCTTATGT | COL4A6 TSS* P1 (human) |
| SEQ ID NO: 51 | CTACCGGCTGCCCAAGGTAG | COL4A6 TSS* P1 (human) |
| SEQ ID NO: 52 | TTGCTTAGGTCTTAGCGTAT | COL4A6 TSS* P1 (human) |
| SEQ ID NO: 53 | GGTACGCACACCAATGAGAT | COL4A6 TSS* P2 (human) |
| SEQ ID NO: 54 | GAGTGACGCTCAGTTATTTG | COL4A6 TSS* P2 (human) |
| SEQ ID NO: 55 | GACTGCACCGGCAACCTGCG | COL4A6 TSS* P2 (human) |
| SEQ ID NO: 56 | GCCGGTGCAGTCTAAAACTG | COL4A6 TSS* P2 (human) |
| SEQ ID NO: 57 | CGGGCGAGTCGCAGCGAGGA | COL5A1 (human) |
| SEQ ID NO: 58 | CCCGGGGCGGAGCGGACGTG | COL5A1 (human) |
| SEQ ID NO: 59 | CCTCGCCCGCGGCGCCCAGT | COL5A1 (human) |
| SEQ ID NO: 60 | CCCCAGGCCCGCCCGCCTAC | COL5A1 (human) |
| SEQ ID NO: 61 | CCAAGCAACGGTCTGATTGA | COL5A2 (human) |
| SEQ ID NO: 62 | CAGAGACGCGTGTTCTGATT | COL5A2 (human) |
| SEQ ID NO: 63 | AAAGTTAAAGGGTGTGTGTC | COL5A2 (human) |
| SEQ ID NO: 64 | TTAACTTTTAAGCATAGATG | COL5A2 (human) |
| SEQ ID NO: 65 | ACTGGAACCCTCGAACTCTA | COL5A3 (human) |
| SEQ ID NO: 66 | TGCACCTCCTCCTAATTCTA | COL5A3 (human) |
| SEQ ID NO: 67 | GGAGGCTGAAGTCTTGAATG | COL5A3 (human) |
| SEQ ID NO: 68 | AACAACCCAGCTCCTGGCGC | COL5A3 (human) |
| SEQ ID NO: 69 | GCGGCCGCGCCAAGATGCGC | ADAMTS2 (human) |
| SEQ ID NO: 70 | GCGAAGAGGGAAGCGGGCGG | ADAMTS2 (human) |
| SEQ ID NO: 71 | CGAGCCCGGCACCGCGGCGA | ADAMTS2 (human) |
| SEQ ID NO: 72 | GCAGAGACACCCCGAGGCGG | ADAMTS2 (human) |
| SEQ ID NO: 73 | CGACCCGCGGGGCGCTAATA | ADAMTS3 (human) |
| SEQ ID NO: 74 | CTCCGCCTAGGGCGAGAGGA | ADAMTS3 (human) |
| SEQ ID NO: 75 | CGCGCCCCCCGCACCGTGTC | ADAMTS3 (human) |
| SEQ ID NO: 76 | GTTCGGGCCCCGCATGACGT | ADAMTS3 (human) |
| SEQ ID NO: 77 | GGGGCTCTGTCCCACCAAAA | ADAMTS4 (human) |
| SEQ ID NO: 78 | AAAAAATGGGACTTGCCCAG | ADAMTS4 (human) |
| SEQ ID NO: 79 | ACAGCTGAGGGCTGATTGTG | ADAMTS4 (human) |
| SEQ ID NO: 80 | CTAGCAGCCGAATGGATAAT | ADAMTS4 (human) |
| SEQ ID NO: 81 | GTCCGCGCCTAATCAGATGG | ADAMTS5 (human) |
| SEQ ID NO: 82 | GAGGAGGGTGATCGAGGAAA | ADAMTS5 (human) |
| SEQ ID NO: 83 | GCAAAAGAGGAGGGTGATCG | ADAMTS5 (human) |
| SEQ ID NO: 84 | AGGCGCGGACTGGGAAGGGT | ADAMTS5 (human) |
| SEQ ID NO: 85 | GGGAGGTAGAGGTACAATCG | ADAMTS6 (human) |
| SEQ ID NO: 86 | AGATGTGCCTGGGCTGCGTC | ADAMTS6 (human) |
| SEQ ID NO: 87 | CCCCCTCCACGTGACGACCC | ADAMTS6 (human) |
| SEQ ID NO: 88 | GTCCTGGGTCGTCACGTGGA | ADAMTS6 (human) |
| SEQ ID NO: 89 | GCAGGCCGTGCTCGCCTCAA | ADAMTS7 (human) |
| SEQ ID NO: 90 | GCGCAGGCAGCGCCCCGCAA | ADAMTS7 (human) |
| SEQ ID NO: 91 | CACGGCCTGCGGGGCCGATG | ADAMTS7 (human) |
| SEQ ID NO: 92 | AGGGGGGCGGACATCCGTTG | ADAMTS7 (human) |
| SEQ ID NO: 93 | CACCCCGGGAAGCACCGAGT | ADAMTS8 (human) |
| SEQ ID NO: 94 | GATCCCAGGGGAGGGGAAAC | ADAMTS8 (human) |
| SEQ ID NO: 95 | GAAAGCGGTTGGGGTCTCCC | ADAMTS8 (human) |
| SEQ ID NO: 96 | TCCTCTGCGGCCAAGAGTCC | ADAMTS8 (human) |
| SEQ ID NO: 97 | GAGGAAAAAGAGACTCGGAA | ADAMTS9 (human) |
| SEQ ID NO: 98 | GCCGAGGAGGGGACATGGTC | ADAMTS9 (human) |
| SEQ ID NO: 99 | CCCCGGTGCACGCCTCTAAG | ADAMTS9 (human) |
| SEQ ID NO: 100 | GAGGCCCTGCCGGCTGCAAG | ADAMTS9 (human) |
| SEQ ID NO: 101 | GGCGGGGCGGCCCGAGTTCC | ADAMTS10 (human) |
| SEQ ID NO: 102 | GCGCCCCCGCCGCGTGGGAG | ADAMTS10 (human) |
| SEQ ID NO: 103 | AGCCAGGGACCCGGGAACTC | ADAMTS10 (human) |
| SEQ ID NO: 104 | GGGCGGGGAGGGGACGAAGC | ADAMTS10 (human) |
| SEQ ID NO: 105 | GAGGCCGCGGGGCATGCGGG | ADAMTS12 (human) |
| SEQ ID NO: 106 | GACAGTGTCCGCTTTCGGCG | ADAMTS12 (human) |
| SEQ ID NO: 107 | CTGGCCGAGCCGGCCAAATA | ADAMTS12 (human) |
| SEQ ID NO: 108 | CCGGCCAAATAGGGGAGACC | ADAMTS12 (human) |
| SEQ ID NO: 109 | GTGCACGCCCACCCCCTTAG | ADAMTS13 (human) |
| SEQ ID NO: 110 | TCTAAGATGGTTGCAGTTCA | ADAMTS13 (human) |
| SEQ ID NO: 111 | GGGAGCGGGGACCCCGGAGA | ADAMTS13 (human) |
| SEQ ID NO: 112 | GCAGGGGCCGTGGCTAGGGT | ADAMTS13 (human) |
| SEQ ID NO: 113 | CAGGCCCTGCGGCCAAGAAA | ADAMTS14 (human) |
| SEQ ID NO: 114 | GGGGATCCCAGGCGTCTGAG | ADAMTS14 (human) |
| SEQ ID NO: 115 | CCGCCCGCGCAGTGACGCTC | ADAMTS14 (human) |
| SEQ ID NO: 116 | GGCTTTGGGCCACCACTCCG | ADAMTS14 (human) |
| SEQ ID NO: 117 | GTGGCTCTCCGCTCTGGAGG | ADAMTS15 (human) |
| SEQ ID NO: 118 | GGAGAGCCACTTGGCGGGGA | ADAMTS15 (human) |
| SEQ ID NO: 119 | GCCACTCCCTCCCCGCCAAG | ADAMTS15 (human) |
| SEQ ID NO: 120 | CAGAGCGGAGAGCCACTTGG | ADAMTS15 (human) |
| SEQ ID NO: 121 | GCCCCTGTGGTCAACCTCGT | ADAMTS16 TSS* P1 (human) |
| SEQ ID NO: 122 | TCGGAATTCCGAGAAGAATC | ADAMTS16 TSS* P1 (human) |
| SEQ ID NO: 123 | CTGCCTACGAGGTTGACCAC | ADAMTS16 TSS* P1 (human) |
| SEQ ID NO: 124 | GGGACACGTAATCGCCCCTG | ADAMTS16 TSS* P1 (human) |
| SEQ ID NO: 125 | GGGAGGAGGCGAGGTCAGCG | ADAMTS16 TSS* P2 (human) |
| SEQ ID NO: 126 | AGGTCAGCGGGGCGCTGAGG | ADAMTS16 TSS* P2 (human) |
| SEQ ID NO: 127 | GACCGAGGAGGGGAGAGTGC | ADAMTS16 TSS* P2 (human) |
| SEQ ID NO: 128 | CCGCGCGGGGACCACACAGT | ADAMTS16 TSS* P2 (human) |
| SEQ ID NO: 129 | GGCGGGGCGGAGGAAAGCGA | ADAMTS17 (human) |
| SEQ ID NO: 130 | TCGACCCATCCCAACCAGTA | ADAMTS17 (human) |
| SEQ ID NO: 131 | GACAACCTAACTACGCACTG | ADAMTS17 (human) |
| SEQ ID NO: 132 | TTCCCCTCGGAAGGCGGGAG | ADAMTS17 (human) |
| SEQ ID NO: 133 | GAGGCTGGGTGGGAGTGATA | ADAMTS18 (human) |
| SEQ ID NO: 134 | AGGGACCGACCGCCATGAGA | ADAMTS18 (human) |
| SEQ ID NO: 135 | CCGGGCTGGTGCAAGGGCGT | ADAMTS18 (human) |
| SEQ ID NO: 136 | GTGCAAGGGCGTGGGATTCC | ADAMTS18 (human) |
| SEQ ID NO: 137 | TAATTCGCCAGGGAGCTCGA | ADAMTS19 (human) |
| SEQ ID NO: 138 | GAGGGAATGAGTAGGGAGAT | ADAMTS19 (human) |
| SEQ ID NO: 139 | GGCTGTGGGTCTGTCTTGTG | ADAMTS19 (human) |
| SEQ ID NO: 140 | GGCGCGGGAGGGAATGAGTA | ADAMTS19 (human) |
| SEQ ID NO: 141 | AGGGTACCGCAGTCCCCTTG | ADAMTS20 (human) |
| SEQ ID NO: 142 | TGCGGCTGAGTGGAGAAGGG | ADAMTS20 (human) |
| SEQ ID NO: 143 | TTGGCCGGGAACAGCCCATA | ADAMTS20 (human) |
| SEQ ID NO: 144 | GCGCGGGGAAAAGCGAGCAG | ADAMTS20 (human) |
| SEQ ID NO: 145 | GTAAGCAGGATGCAAGTGAT | TLL1 (human) |
| SEQ ID NO: 146 | TTGGCTAGGGGCCCACGGGT | TLL1 (human) |
| SEQ ID NO: 147 | GAGCAAAAGTGTGAGGATTT | TLL1 (human) |
| SEQ ID NO: 148 | GGGGAGGGCGCAGGCAAACT | TLL1 (human) |
| SEQ ID NO: 149 | CGGCACCTTCGCAACTTCGG | TLL2 (human) |
| SEQ ID NO: 150 | GCCTCGGGCGCCCGAGTGAT | TLL2 (human) |
| SEQ ID NO: 151 | TGCTCCTGAAGATCGGGACT | TLL2 (human) |
| SEQ ID NO: 152 | GCGCCCCGGGCAGGCGCCTT | TLL2 (human) |
| SEQ ID NO: 153 | CCCGCCCCCCGCTCGGTCCG | BMP1 (human) |
| SEQ ID NO: 154 | CGGAGCCGCGGACCGAGCGG | BMP1 (human) |
| SEQ ID NO: 155 | GGCCCGGCCGAGCACTGTCC | BMP1 (human) |
| SEQ ID NO: 156 | CACTGTCCCGGCCCCGAGGG | BMP1 (human) |

*TSS = Transcription Start Site Promoter 1 (P1) or P2

TABLE 4
Genomic Locations and PAMs of crRNA Sequences

| | Genomic Location | PAM |
|---|---|---|
| SEQ ID NO: 1 | hg38:17:50201809-50201828:− | GGG |
| SEQ ID NO: 2 | hg38:17:50201820-50201839:− | AGG |
| SEQ ID NO: 3 | hg38:17:50201748-50201767:+ | TGG |
| SEQ ID NO: 4 | hg38:17:50201683-50201702:+ | GGG |
| SEQ ID NO: 5 | hg38:7:94394807-94394826:− | GGG |
| SEQ ID NO: 6 | hg38:7:94394736-94394755:− | GGG |
| SEQ ID NO: 7 | hg38:7:94394716-94394735:− | GGG |
| SEQ ID NO: 8 | hg38:7:94394596-94394615:+ | AGG |
| SEQ ID NO: 9 | hg38:14:75982087-75982106:− | AGG |
| SEQ ID NO: 10 | hg38:14:75982399-75982418:+ | GGG |
| SEQ ID NO: 11 | hg38:14:75982074-75982093:− | TGG |
| SEQ ID NO: 12 | hg38:14:75982338-75982357:− | GGG |
| SEQ ID NO: 13 | hg38:19:41354142-41354161:+ | CGG |
| SEQ ID NO: 14 | hg38:19:41354044-41354063:− | AGG |
| SEQ ID NO: 15 | hg38:19:41353785-41353804:+ | CGG |
| SEQ ID NO: 16 | hg38:19:41354040-41354059:− | CGG |
| SEQ ID NO: 17 | hg38:1:218345204-218345223:− | AGG |
| SEQ ID NO: 18 | hg38:1:218345171-218345190:− | GGG |
| SEQ ID NO: 19 | hg38:1:218345177-218345196:− | GGG |
| SEQ ID NO: 20 | hg38:1:218345221-218345240:+ | AGG |
| SEQ ID NO: 21 | hg38:12:48004733-48004752:− | GGG |
| SEQ ID NO: 22 | hg38:12:48004679-48004698:− | TGG |
| SEQ ID NO: 23 | hg38:12:48004682-48004701:− | GGG |
| SEQ ID NO: 24 | hg38:12:48004634-48004653:+ | TGG |
| SEQ ID NO: 25 | hg38:2:188974289-188974308:+ | GGG |
| SEQ ID NO: 26 | hg38:2:188974039-188974058:+ | GGG |
| SEQ ID NO: 27 | hg38:2:188974220-188974239:+ | AGG |
| SEQ ID NO: 28 | hg38:2:188973838-188973857:+ | GGG |
| SEQ ID NO: 29 | hg38:13:110307345-110307364:− | CGG |
| SEQ ID NO: 30 | hg38:13:110307240-110307259:+ | AGG |
| SEQ ID NO: 31 | hg38:13:110307269-110307288:+ | TGG |
| SEQ ID NO: 32 | hg38:13:110307283-110307302:− | CGG |
| SEQ ID NO: 33 | hg38:13:110307127-110307146:− | CGG |
| SEQ ID NO: 34 | hg38:13:110307129-110307148:+ | CGG |
| SEQ ID NO: 35 | hg38:13:110307080-110307099:+ | AGG |
| SEQ ID NO: 36 | hg38:13:110307021-110307040:+ | AGG |
| SEQ ID NO: 37 | hg38:2:227164435-227164454:− | GGG |
| SEQ ID NO: 38 | hg38:2:227164448-227164467:− | AGG |
| SEQ ID NO: 39 | hg38:2:227164449-227164468:+ | GGG |
| SEQ ID NO: 40 | hg38:2:227164468-227164487:+ | TGG |
| SEQ ID NO: 41 | hg38:2:227164435-227164454:− | GGG |
| SEQ ID NO: 42 | hg38:2:227164448-227164467:− | AGG |
| SEQ ID NO: 43 | hg38:2:227164329-227164348:− | AGG |
| SEQ ID NO: 44 | hg38:2:227164385-227164404:− | AGG |
| SEQ ID NO: 45 | hg38:X:108439713-108439732:− | GGG |
| SEQ ID NO: 46 | hg38:X:108439736-108439755:− | AGG |
| SEQ ID NO: 47 | hg38:X:108439581-108439600:+ | TGG |
| SEQ ID NO: 48 | hg38:X:108439726-108439745:+ | CGG |
| SEQ ID NO: 49 | hg38:X:108438581-108438600:− | GGG |
| SEQ ID NO: 50 | hg38:X:108438679-108438698:− | GGG |
| SEQ ID NO: 51 | hg38:X:108438493-108438512:− | GGG |
| SEQ ID NO: 52 | hg38:X:108438592-108438611:− | AGG |
| SEQ ID NO: 53 | hg38:X:108439581-108439600:+ | TGG |
| SEQ ID NO: 54 | hg38:X:108439713-108439732:− | GGG |
| SEQ ID NO: 55 | hg38:X:108439736-108439755:− | AGG |
| SEQ ID NO: 56 | hg38:X:108439744-108439763:+ | TGG |
| SEQ ID NO: 57 | hg38:9:134641608-134641627:− | AGG |
| SEQ ID NO: 58 | hg38:9:134641579-134641598:− | GGG |
| SEQ ID NO: 59 | hg38:9:134641640-134641659:+ | GGG |
| SEQ ID NO: 60 | hg38:9:134641522-134641541:+ | CGG |
| SEQ ID NO: 61 | hg38:2:189179867-189179886:+ | TGG |
| SEQ ID NO: 62 | hg38:2:189179900-189179919:− | TGG |
| SEQ ID NO: 63 | hg38:2:189180069-189180088:− | TGG |
| SEQ ID NO: 64 | hg38:2:189179943-189179962:+ | GGG |
| SEQ ID NO: 65 | hg38:19:10010713-10010732:− | GGG |
| SEQ ID NO: 66 | hg38:19:10010816-10010835:− | GGG |
| SEQ ID NO: 67 | hg38:19:10010786-10010805:− | AGG |
| SEQ ID NO: 68 | hg38:19:10010646-10010665:− | TGG |
| SEQ ID NO: 69 | hg38:5:179345605-179345624:− | CGG |
| SEQ ID NO: 70 | hg38:5:179345630-179345649:− | CGG |
| SEQ ID NO: 71 | hg38:5:179345657-179345676:− | CGG |
| SEQ ID NO: 72 | hg38:5:179345684-179345703:+ | CGG |
| SEQ ID NO: 73 | hg38:4:72569368-72569387:+ | AGG |
| SEQ ID NO: 74 | hg38:4:72569539-72569558:+ | GGG |
| SEQ ID NO: 75 | hg38:4:72569453-72569472:+ | AGG |
| SEQ ID NO: 76 | hg38:4:72569432-72569451:− | GGG |
| SEQ ID NO: 77 | hg38:1:161199300-161199319:− | AGG |
| SEQ ID NO: 78 | hg38:1: 161199176-161199195:+ | GGG |
| SEQ ID NO: 79 | hg38:1:161199270-161199289:− | GGG |
| SEQ ID NO: 80 | hg38:1:161199209-161199228:+ | AGG |
| SEQ ID NO: 81 | hg38:21:26967216-26967235:+ | GGG |
| SEQ ID NO: 82 | hg38:21:26967248-26967267:+ | GGG |
| SEQ ID NO: 83 | hg38:21:26967242-26967261:+ | AGG |
| SEQ ID NO: 84 | hg38:21:26967206-26967225:− | GGG |
| SEQ ID NO: 85 | hg38:5:65481993-65482012:+ | GGG |
| SEQ ID NO: 86 | hg38:5:65481934-65481953:+ | GGG |
| SEQ ID NO: 87 | hg38:5:65481971-65481990:− | AGG |
| SEQ ID NO: 88 | hg38:5:65481966-65481985:+ | GGG |
| SEQ ID NO: 89 | hg38:15:78811581-78811600:+ | GGG |
| SEQ ID NO: 90 | hg38:15:78811616-78811635:+ | CGG |
| SEQ ID NO: 91 | hg38 15:78811571-78811590:− | AGG |
| SEQ ID NO: 92 | hg38:15:78811633-78811652:− | CGG |
| SEQ ID NO: 93 | hg38:11:130428594-130428613:+ | GGG |
| SEQ ID NO: 94 | hg38:11:130428692-130428711:− | GGG |
| SEQ ID NO: 95 | hg38:11:130428906-130428925:− | AGG |
| SEQ ID NO: 96 | hg38:11:130428926-130428945:+ | AGG |
| SEQ ID NO: 97 | hg38:3:64688410-64688429:− | GGG |
| SEQ ID NO: 98 | hg38:3:64688158-64688177:− | TGG |
| SEQ ID NO: 99 | hg38:3:64688088-64688107:+ | AGG |
| SEQ ID NO: 100 | hg38:3:64688202-64688221:+ | AGG |
| SEQ ID NO: 101 | hg38:19:8610882-8610901:− | CGG |
| SEQ ID NO: 102 | hg38: 19:8610910-8610929:+ | GGG |
| SEQ ID NO: 103 | hg38: 19:8610869-8610888:+ | GGG |
| SEQ ID NO: 104 | hg38:19:8610949-8610968:+ | GGG |
| SEQ ID NO: 105 | hg38:5:33892158-33892177:− | AGG |
| SEQ ID NO: 106 | hg38:5:33892078-33892097:+ | GGG |
| SEQ ID NO: 107 | hg38:5:33892259-33892278:+ | GGG |
| SEQ ID NO: 108 | hg38:5:33892268-33892287:+ | CGG |
| SEQ ID NO: 109 | hg38:9:133422154-133422173:− | AGG |
| SEQ ID NO: 110 | hg38:9:133422219-133422238:− | GGG |
| SEQ ID NO: 111 | hg38:9:133422039-133422058:+ | GGG |
| SEQ ID NO: 112 | hg38:9:133422279-133422298:− | GGG |
| SEQ ID NO: 113 | hg38:10:70672206-70672225:+ | GGG |
| SEQ ID NO: 114 | hg38:10:70672269-70672288:+ | GGG |
| SEQ ID NO: 115 | hg38:10:70672499-70672518:− | CGG |
| SEQ ID NO: 116 | hg38:10:70672250-70672269:+ | GGG |
| SEQ ID NO: 117 | hg38:11:130448484-130448503:+ | CGG |
| SEQ ID NO: 118 | hg38:11:130448474-130448493:− | GGG |
| SEQ ID NO: 119 | hg38:11:130448465-130448484:+ | TGG |
| SEQ ID NO: 120 | hg38:11:130448480-130448499:− | CGG |
| SEQ ID NO: 121 | hg38:5:5146151-5146170:− | AGG |
| SEQ ID NO: 122 | hg38:5:5146075-5146094:− | AGG |
| SEQ ID NO: 123 | hg38:5:5146145-5146164:+ | AGG |
| SEQ ID NO: 124 | hg38:5:5146164-5146183:− | TGG |
| SEQ ID NO: 125 | hg38:5:5140149-5140168:− | GGG |
| SEQ ID NO: 126 | hg38:5:5140138-5140157:− | CGG |
| SEQ ID NO: 127 | hg38:5:5140186-5140205:− | AGG |
| SEQ ID NO: 128 | hg38:5:5140010-5140029:− | TGG |
| SEQ ID NO: 129 | hg38:15:100342081-100342100:− | GGG |
| SEQ ID NO: 130 | hg38:15:100342112-100342131:− | GGG |
| SEQ ID NO: 131 | hg38:15:100342137-100342156:− | TGG |
| SEQ ID NO: 132 | hg38:15:100342049-100342068:+ | GGG |
| SEQ ID NO: 133 | hg38:16:77435213-77435232:+ | GGG |
| SEQ ID NO: 134 | hg38:16:77435240-77435259:+ | AGG |
| SEQ ID NO: 135 | hg38:16:77435148-77435167:+ | GGG |
| SEQ ID NO: 136 | hg38:16:77435156-77435175:+ | CGG |
| SEQ ID NO: 137 | hg38:5:129460117-129460136:+ | GGG |
| SEQ ID NO: 138 | hg38:5:129459790-129459809:− | GGG |
| SEQ ID NO: 139 | hg38:5:129460024-129460043:+ | GGG |
| SEQ ID NO: 140 | hg38:5:129459797-129459816:− | GGG |
| SEQ ID NO: 141 | hg38:12:43552359-43552378:− | CGG |
| SEQ ID NO: 142 | hg38:12:43552341-43552360:− | AGG |
| SEQ ID NO: 143 | hg38:12:43552274-43552293:− | GGG |
| SEQ ID NO: 144 | hg38:12:43552313-43552332:− | GGG |
| SEQ ID NO: 145 | hg38:4:165873143-165873162:+ | TGG |
| SEQ ID NO: 146 | hg38:4:165873672-165873691:− | GGG |
| SEQ ID NO: 147 | hg38:4:165873253-165873272:− | GGG |
| SEQ ID NO: 148 | hg38:4:165873615-165873634:− | CGG |
| SEQ ID NO: 149 | hg38:10:96514056-96514075:+ | AGG |
| SEQ ID NO: 150 | hg38:10:96514019-96514038:− | TGG |
| SEQ ID NO: 151 | hg38:10:96514190-96514209:+ | GGG |
| SEQ ID NO: 152 | hg38:10:96514142-96514161:− | AGG |
| SEQ ID NO: 153 | hg38:8:22165233-22165252:− | CGG |
| SEQ ID NO: 154 | hg38:8:22165225-22165244:+ | GGG |
| SEQ ID NO: 155 | hg38:8:22165045-22165064:+ | CGG |
| SEQ ID NO: 156 | hg38:8:22165057-22165076:+ | GGG |

The DharmaFECT Duol Transfection Reagent has been shown to be an efficient transfection reagent for transfection of small RNAs and plasmids simultaneously (Borawski, et al., 2007).

An example of a dCas9 protein for use with the present technology is one having the amino acid sequence shown below (SEQ ID NO: 157 (uniprot.org/uniprot/AOA386IRG9)):

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNEDK
NLPNEKVLPKHSLLYEYFTVYNELTKVKYVTEGMRKPAFLSGEQKKAIVD
LLFKTNRKVTVKQLKEDYFKKIECFDSVEISGVEDRENASLGTYHDLLKI
IKDKDELDNEENEDILEDIVLTLTLFEDREMIEERLKTYAHLEDDKVMKQ
LKRRRYTGWGRLSRKLINGIRDKQSGKTILDELKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKEDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLETLTNLGAPAAFKYFDTTIDRKRYTSTKEVLDATLIHQ
SITGLYETRIDLSQLGGD

An example of a synthetic tracrRNA for use with the present technology is one published by Jinek, et al., (2012), which has the sequence GGAACCAUUCAAAACAGCAUAGCAAGUUAAAAUAAGGCUAGUCCGUUAUCAACUUGA AAAAGUGGCACCGAGUCGGUGCUUUUUUU (SEQ ID NO:158). The crRNA may include a region complementary to a portion of the tracrRNA. Alternatively, a linker sequence can be added between the crRNA and the tracrRNA to yield a single gRNA molecule.

VP64 is a transcriptional activator including four tandem copies of VP16 (Herpes Simplex Viral Protein 16, amino acids 437-447, connected with glycine-serine linkers). The amino acid sequence of VP64 is shown below (SEQ ID NO: 159, (parts.igem.org/Part:BBa_J176013)):

GACGCTTTGGACGACTTCGACTTGGACATGTTGGGTTCTGACGCTTTGGA
CGACTTCGACTTGGACATGTTGGGTTCTGACGCTTTGGACGACTTCGACT
TGGACATGTTGGGTTCTGACGCTTTGGACGACTTCGACTTGGACATGTTG

The transcriptional activator β65 includes four isoforms produced by alternative splicing (uniprot.org/uniprot/Q04206). Isoform 1 has the amino acid sequence shown below (SEQ ID NO: 160).

MDELFPLIFPAEPAQASGPYVEIIEQPKQRGMRFRYKCEGRSAGSIPGER
STDTTKTHPTIKINGYTGPGTVRISLVTKDPPHRPHPHELVGKDCRDGFY
EAELCPDRCIHSFQNLGIQCVKKRDLEQAISQRIQTNNNPFQVPIEEQRG
DYDLNAVRLCFQVTVRDPSGRPLRLPPVLSHPIFDNRAPNTAELKICRVN
RNSGSCLGGDEIFLLCDKVQKEDIEVYFTGPGWEARGSFSQADVHRQVAI
VFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYP
FTSSLSTINYDEFPTMVFPSGQISQASALAPAPPQVLPQAPAPAPAPAMV
SALAQAPAPVPVLAPGPPQAVAPPAPKPTQAGEGTLSEALLQLQFDDEDL
GALLGNSTDPAVFTDLASVDNSEFQQLLNQGIPVAPHTTEPMLMEYPEAI
TRLVTGAQRPPDPAPAPLGAPGLPNGLLSGDEDESSIADMDESALLSQIS
S

The amino acid sequence of the transcriptional activator HSF1, SEQ ID NO: 161, is shown below (SEQ ID NO: 161 ((uniprot.org/uniprot/Q00613)).

MDLPVGPGAAGPSNVPAFLTKLWTLVSDPDTDALICWSPSGNSFHVEDQG
QFAKEVLPKYFKHNNMASFVRQLNMYGFRKVVHIEQGGLVKPERDDTEFQ
HPCFLRGQEQLLENIKRKVTSVSTLKSEDIKIRQDSVTKLLTDVQLMKGK
QECMDSKLLAMKHENEALWREVASLRQKHAQQQKVVNKLIQFLISLVQSN
RILGVKRKIPLMLNDSGSAHSMPKYSRQFSLEHVHGSGPYSAPSPAYSSS
SLYAPDAVASSGPIISDITELAPASPMASPGGSIDERPLSSSPLVRVKEE
PPSPPQSPRVEEASPGRPSSVDTLLSPTALIDSILRESEPAPASVTALTD
ARGHTDTEGRPPSPPPTSTPEKCLSVACLDKNELSDHLDAMDSNLDNLQT
MLSSHGFSVDTSALLDLFSPSVTVPDMSLPDLDSSLASIQELLSPQEPPR
PPEAENSSPDSGKQLVHYTAQPLFLLDPGSVDTGSNDLPVLFELGEGSYF
SEGDGFAEDPTISLLTGSEPPKAKDPTVS

The amino acid sequence of transcriptional activator MS2 is shown below (SEQ ID NO: 162 (uniprot.org/uniprot/P03612)).

MASNFTQFVLVDNGGTGDVTVAPSNFANGVAEWISSNSRSQAYKVTCSVR
QSSAQNRKYTIKVEVPKVATQTVGGVELPVAAWRSYLNMELTIPIFATNS
DCELIVKAMQGLLKDGNPIPSAIAANSGIY

The dCas9 mRNA, described above, is the limiting reagent for the protocol described below, allowing for 11 wells to be made with 5 nmol of starting material. An example protocol for plating the test plate conditions is:
1. Plate fibroblasts in a 48 well plate at a density where the wells will be 70-90 percent confluent the next day.
2. Dilute and mix COL1A1, COL1A2, and TGF-β3 crRNA and tracrRNA (1:1 total crRNA to tracrRNA) to a working concentration of 2.5μM according to Dharmacon's resuspension procedure [165]. This is now the gRNA solution.
   a. When purchased, crRNA and tracrRNA comes as 5nmol. Dilute each of these to 5μM by adding 1000μL of 10 mM Tris pH 7.4 to each stock of crRNA pools and tracrRNA.
   b. When mixed 1:1, the resulting tracrRNA:crRNA should have a working concentration of 2.5μM.
3. Dilute 20pg dCas9 mRNA to a concentration of 100 ng/pL in 200μL serum-free medium.
4. Add 3μL of each of the three gRNA solutions, 18μL of dCas9 solution, and 9 μL of medium (to bring to a volume of 30 μL) in a microcentrifuge tube.
5. The original protocol calls for a testing of a range of transfection reagent amounts (0.1-.8 μL)to determine which amount is best for your cell type. When scaled from a 96 to 48well plate configuration and 3x mRNA amounts (3 different gRNA-Cas9 mRNA complexes, one for each gene of interest), this range is 0.9-7.2 μL of transfection reagent. Generate a range of Duo Transfection reagent working solution amounts consisting of 7.2, 4.05, and 0.9 μL of transfection reagent and bring each of these to a volume of 30 μL with serum-free media.
6. Incubate at room temperature for 5 minutes
7. Combine 30 μL of each concentration of transfection reagent with 30 μL of the dCas9/gRNA solution and mix gently with a pipette.
8. Incubate at room temperature for 20 minutes
9. Add 240 μL serum-free media to each combination and replace media on cells with the newly developed transfection mixtures.
10. Harvest media for collagen assay every 48 hours for 108 hours.
11. Gene activation of COL1A1, COL1A2, and TGFβ3 should start to occur around 24 hours and have maximal expression until about 72 hours after transfection.

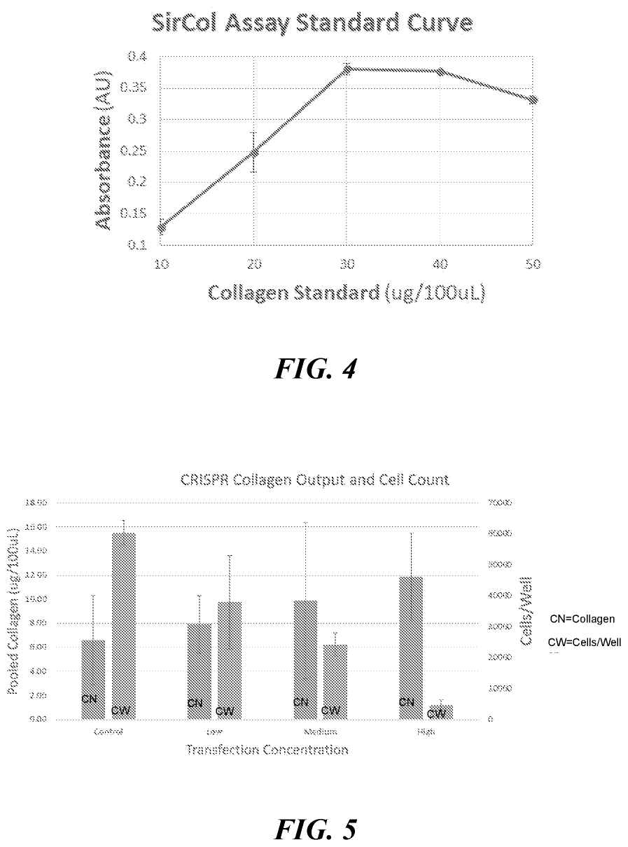

To measure collagen production, a hydroxyproline assay can be used, optionally after concentrating the collagen solution or culture medium (see, e.g., D.D. Cissell et al., (2017). Tissue Eng Part C Methods. 2017 Apr;23(4):243-25) Alternatively, the SirCoI™ dye binding collagen assay can be used (www.biocolor.co.uk/product/sircol-soluble-collagen-assay). A standard curve, as shown in FIG. 4, can be determined using the SirCoI™ Assay procedure with five standard solutions composed of the SirCoI™ collagen standard mixed with DMEM culture medium.

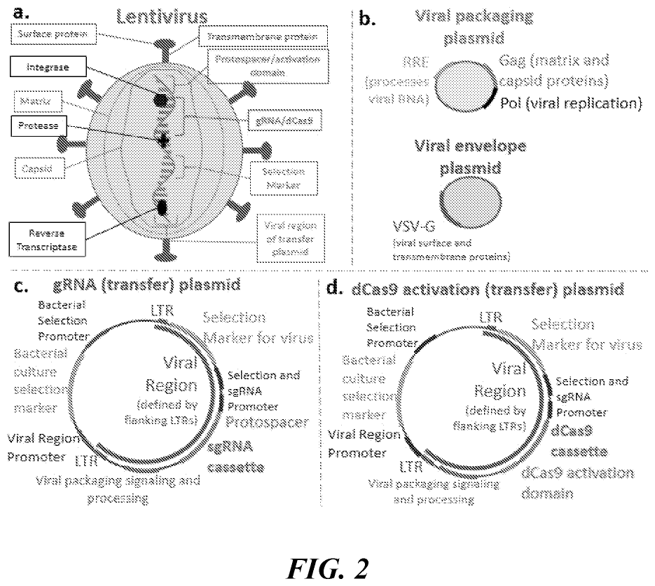

Three levels of transfection reagent were compared; transfection reagents can be toxic to cells at high concentrations. The high (H), medium (M), and low (L), transfection reagent concentrations were 7.2, 4.05, and 0.9 μL of transfection reagent per well, respectively. Media was harvested every 12 hours and pooled in two-day segments. Collagen amount per well was quantified every two days, and cell counts were performed after the end of media harvesting. Collagen produced per cell was estimated based on the number of cells per well at the end of media collection. A comparison of total (days 1-6) pooled collagen production (per well, not per cell) for each concentration level and the cell count is shown in FIG. 2. The figure shows collagen production and cell count bytransfection concentration of high, medium, and low compared to control. Despite cell viability constantly dropping with increased transfection reagent, there was an observed trend (albeit not statistically significant) of increased total collagen per well with increased transfection reagent, despite these wells having less collagen-producing fibroblasts than wells with less transfection reagent. This indicates increased collagen production per cell in the CRISPR cells compared to the control cells.

Table 5 shows mean collagen production (molecules/cell/hours) for the test groups (high, medium, low), and the group names denote the transfection level and the days at which the assay was taken (i.e., 1-2=days 1 and 2).

TABLE 5
Collagen Production of Test Groups.

| | Mean Collagen Production | | |
|---|---|---|---|
| Group | (molecules/cell/hour, millions) | Standard Deviation | n |
| Control 1-2 | 1.06 | 0.00 | 3 |
| Control 3-4 | 4.86 | 3.91 | 3 |
| Control 5-6 | 1.06 | 0.00 | 3 |
| Low 1-2 | 1.06 | 0.00 | 3 |
| Low 3-4 | 6.47 | 6.01 | 3 |
| Low 5-6 | 4.49 | 5.94 | 3 |
| Med 1-2 | 1.06 | 0.00 | 3 |
| Med 3-4 | 6.62 | 5.69 | 3 |
| Med 5-6 | 15.93 | 16.65 | 3 |
| High 1-2 | 1.06 | 0.00 | 3 |
| High 3-4 | 44.29 | 74.88 | 3 |
| High 5-6 | 95.39 | 40.16 | 3 |

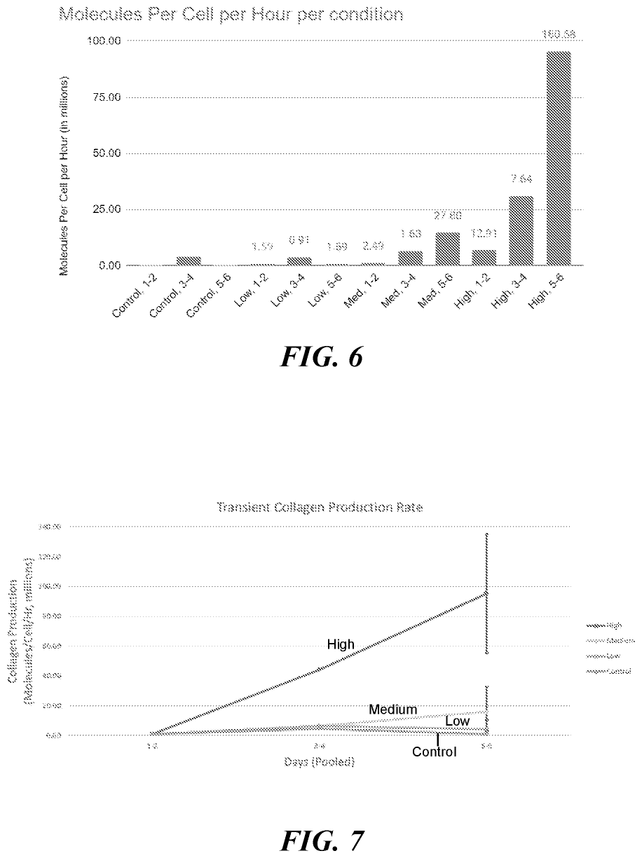

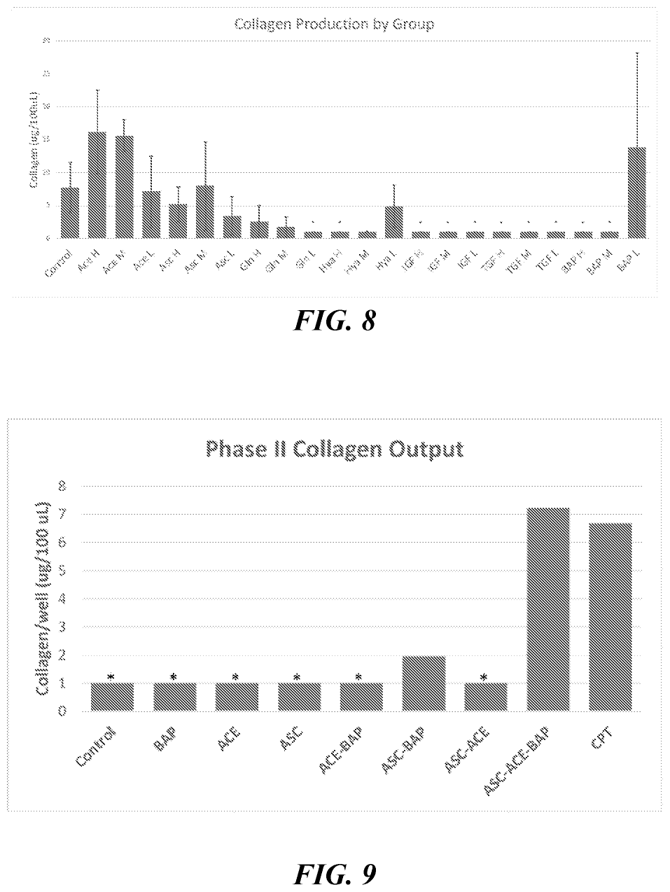

Quantifications that fall below the minimum detectable collagen amount are set to the minimum detectable collagen amount (1.06 million molecules/cell/hour) (Table 5). Having some quantifications fall below the minimum detectable amount is necessary to capture the upper limits of collagen production in the CRISPR conditions. Data for days 1-2 and most control samples were at or below the minimum detectable collagen amount, but every datapoint for CRISPR results at days 3-4 and 5-6 is above the minimum threshold. The fact that the results at days 1-2 matched the control wells is consistent with the expectation that CRISPR should upregulate collagen production after about two days. This increase in collagen is shown in a graphical format in FIG. 6.

Based on the graph in FIG. 6, there are likely differences in collagen production both transiently and due to transfection reagent concentration. In order to determine if the increased collagen per cell in the CRISPR samples was statistically significant, a Tukey Pairwise comparison was performed and is shown in Table 6. Groups that are not in any similar subsets are statistically different at a=0.05 (table generated in IBM SPSS 25; collagen production is in molecules/cell/hours, millions.

TABLE 6
Tukey Pairwise Comparison Chart for Table 5 and FIG. 6.

Subset

| Group | N | 1 | 2 |
|---|---|---|---|
| Control 1-2 | 3 | 1.0600 | |
| Control 5-6 | 3 | 1.0600 | |
| High 1-2 | 3 | 1.0600 | |
| Low 1-2 | 3 | 1.0600 | |
| Med 1-2 | 3 | 1.0600 | |
| Low 5-6 | 3 | 4.4900 | |
| Control 3-4 | 3 | 4.8633 | |
| Low 3-4 | 3 | 6.4667 | |
| Med 3-4 | 3 | 6.6167 | |
| Med 5-6 | 3 | 15.9333 | |
| High 3-4 | 3 | 44.2933 | 44.2933 |
| High 5-6 | 3 | | 95.3867 |

Table 6 shows the subset groupings for the experiment represented in FIG. 6, and Table 5. High transfection concentration at days 5-6 shows a statistically significant increase in collagen production at 0.05 significance. Given the sample sizes and variances, the test has an observed power of 0.971 and an effect size of 1.30. This can be seen in a time plot in the graph shown in FIG. 7, which shows transient collagen production rate over a six-day period. In FIG. 7, error bars are shown for collagen production during days 5-6 (conditions in other days do not show a statistically significant difference).

In order to prevent undesired crosslinking of newly synthesized collagen, BAPN is preferably added to the culture medium. Culture conditions may also be adjusted to optimize preservation of collagen structure and function. For example, since collagen is unstable if stored at 37° C., cells can be cultured below 37° C., and/or collagen can be harvested periodically (e.g., every 8-24 hours), and then stored at a lower temperature to preserve stability.

From the data in Table 5 and FIG. 6 it was shown that the CRISPR system can increase collagen production about 90-fold (about 40% std) compared to the experimental control.

As discussed above, it was found that the CRISPR system can increase collagen production about 90.29-fold in collagen production compared to the experimental control. When scaled up to a T-75 flask, this production level would yield about 554 mg collagen/week. However, the purchasing of commercially available dCas9 mRNA, gRNA (crRNA and tracrRNA) reagents for each gene of interest, and transfection reagent, would be cost prohibitive. Therefore, it is desirable to scale up using other methods of CRISPRa delivery that would cost significantly less. Suitable methods include the use of bacteria to produce dCas9 and gRNA plasmids and viral vectors produced by Human Embryonic kidney (HEK) cells to deliver and stably integrate the dCas9 and gRNA sequences into the host genome would significantly reduce the cost of scale-up and result in lower collagen production costs.

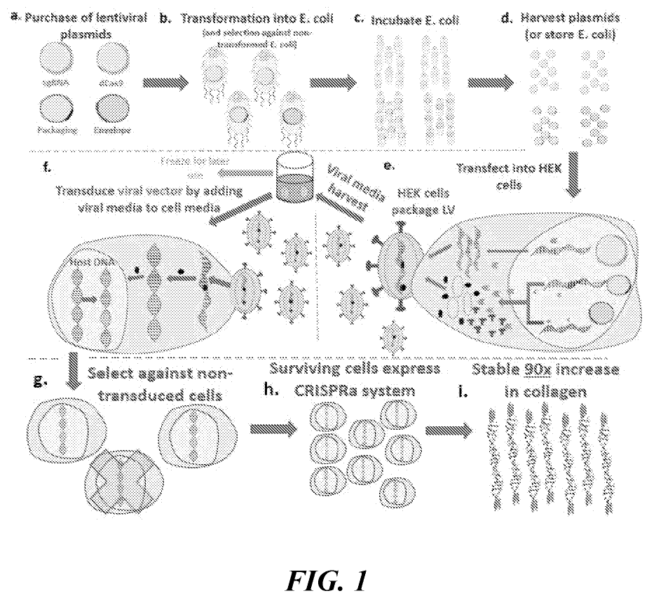

A CRISPRa process involving transduction of cells using a lentiviral vector is illustrated in FIG. 1. First, three types of lentiviral plasmids are purchased (or engineered): transfer, packaging, and envelope (FIG. 1, panel A). Transfer plasmids contain the transgenes to be integrated in the host cells (dCas9 and/or gRNA) and packaging and envelope plasmids contain the components needed to build a lentivirus. The components of a lentivirus and the plasmids needed to make them are detailed in an example below. These plasmids are then transformed into E. coli (FIG. 1, panel B), replicated by incubation (FIG. 1, panel C), and harvested (FIG. 1, panel D). These plasmids are then transfected into HEK cells. HEK cells then assemble the virus and excrete it into the media (FIG. 1, panel E). This viral media is then added to host cells (fibroblasts). The LVs in the viral media then integrate the transgene (dCas9 and/or gRNA) into the host cell (FIG. 1, panel F). These cells are then exposed to antibiotics that kill any non-transduced cells (FIG. 1, panel G). Transduced cells survive due to an antibiotic resistance that is a part of the transgene vector. Surviving cells expressing the transgene (dCas9 and/or gRNA) are then multiplied and used for collagen harvesting (FIG. 1, panel H). A stable increase in collagen is provided (FIG. 1, panel I). Further, overexpression of CRISPR-targeted collagen-related genes is stable and conserved with cell growth and passaging.

For transduction, once the virus production pipeline is established, viral plasmids are cheaply generated by bacteria, transfected into HEK cells that readily package and excrete a supply of viral particles, and added to host cell media for integration of gene of interest into host cell's genome.

The first decision to make for scaling up the collagen production process with viral delivery is deciding what kind of virus to use. Briefly, the benefits of using AAVs (adeno-associated viruses) include high titers, versatility from the availability of multiple serotypes that target different cell types, low toxicity as the virus remains in the episome or in a specific locus on chromosome 19, and low immunogenicity as there is minimal host immune response. The benefits of LVs include that they infect nearly all mammalian cell types, they can be used to deliver relatively large DNA sequences-usually about 5-6 kb in length, and they can be used to generate stable cell lines or drive stable gene expression in organs and tissues in vivo due to integration of the transgene at random locations in the genome. Because the LVs allow for quick and easy stable integration of transgenes, they are a clear choice here, especially because the same class of LV could be used for any cell type that is chosen to work with (epidermal fibroblasts, corneal fibroblasts, iPSCs, etc.). AAVs might be considered if there was a desire to develop a system to be used in a patient that targets a specific tissue type for increased collagen production. The details of the components that make up a LV and how plasmids can be used to construct LVs are further detailed herein in the example shown in FIG. 2.

There are three main genes involved in lentivirus construction: env, gag, and pol. Env, or recombinant VSV-G, (FIG. 2, panel B) encodes a surface glycoprotein that is cut into two subunits: a surface protein and a transmembrane protein (oval and rectangle, respectively, in FIG. 2, panel A). These proteins are essential for virus recognition of, adherence to, and entry into host cells. VSV-G is widely used, as opposed to wild type env, because it provides a more stable glycoprotein that recognizes a wider subset of cell types. Gag (FIG. 2, panel B) is transcribed and spliced into matrix, capsid (light dashed and solid lines, respectively, in FIG. 2, panel A), and nucleocapsid proteins. Matrix proteins are involved in viral assembly. Capsid proteins form the hydrophobic core of the virus, and nucleocapsid proteins coat and protect the transgene. Pol (thicker black line vector indicated in FIG. 2, panel B) encodes three proteins essential for viral replication: protease, reverse transcriptase, and integrase (black oval, plus, and hexagon in FIG. 2, panel A). Protease plays a role in polyprotein precursor processing and virus maturation. Reverse transcriptase is used to turn the RNA vector of interest delivered by the virus into DNA upon delivery into host cells. Integrase is used to integrate the new DNA transgene into the host genome. Once in the host genome, the long terminal repeats (LTR) region of the transgene acts as a ubiquitous promoter and enhancer to ensure expression in the host cell. In wild type HIV, viral proteins (vif, vpr, vpn and nef) are included in the transgene that, at this stage, are transcribed and translated to manufacture new viruses. These proteins have been eliminated from current lentivirus systems in order to ensure that infected cells do not become pathogenic and infect other cells (for operator safety reasons). Another important component of viral processing is RRE. RRE acts to facilitate viral RNA transport during viral packaging (RRE in FIG. 2, panel B). Together, these components act to assemble a lentivirus capable of delivering a vector of interest (transgene).

The last component is the transfer plasmid, which carries the vector of interest to be integrated into a host cell. This vector is usually engineered with restriction enzymes before being inserted into a bacterial cell for replication. As such, the transfer plasmid has a viral region with the vector of interest (and some other needed components) and a non-viral region with an antibiotic resistance gene. This antibiotic resistance is used for selection against bacteria without the new engineered plasmid following transformation (plasmid insertion). The viral region is set off by the aforementioned LTRs. In addition to acting as a promoter once in the host genome, these LTRs on either side of the vector of interest allow the vector to be recognized by multiple other viral proteins in the virus production and transduction process.

Once in the host genome, transcription of this vector has two main functions. The first is to select against cells that have not been transduced. In the transgene there is an antibiotic resistance gene that is transcribed by host cells (FIG. 2, panels C and D). This allows for selection of non-transduced cells. Finally, this transgene also carries the construct that is intended to be transcribed by the host cell (in this case either dCas9 or gRNA) (FIG. 2, panels C and D).

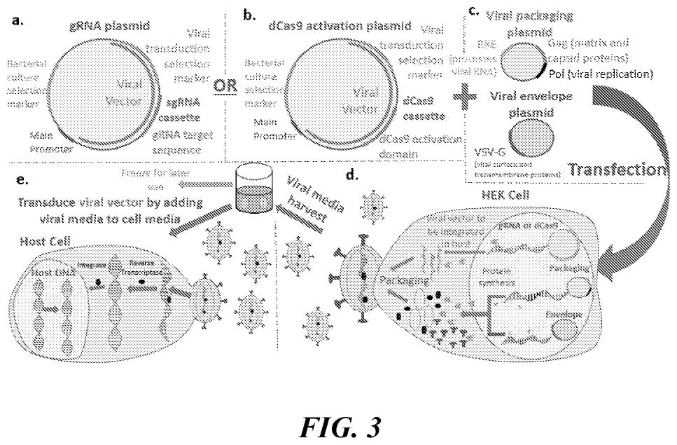

FIG. 3 describes LV packaging and transduction of host cells. First, lentivirus-compatible plasmids, called transfer plasmids, containing the vector of interest (gRNA or dCas9) are cultured in bacteria (FIG. 3, panels A and B). These plasmids are then harvested and mixed with lentivirus envelope and packaging plasmids (FIG. 3, panel C). These plasmids contain the viral components needed to build a virus that delivers the vector of interest. These plasmids are then mixed with transfection reagent and added to the media of HEK cells. Over a span of about 24 hours, the HEK cells will transcribe and/or translate the plasmids into the RNA/proteins needed to make the viruses, and then the HEK cells will use these new RNA and proteins to package the viruses and secrete them into the media (FIG. 3, panel D). This media can then either be used immediately, or it can be frozen and stored for later use. The harvested viral media is then added to the media of the cells of interest (FIG. 3, panel E). After the addition of viral media, the lentivirus deposits the vector RNA of interest into the cell, continually duplicates this RNA into DNA using reverse transcriptase packaged with the virus, and integrates this new DNA into the host cell's genome using integrase packaged with the virus. Reverse transcriptase and integrase are represented as the black dot, for example in FIG. 3, panel E. FIG. 3 shows an example of viral proteins involved in this process. Lastly, transduced cells can then be selected for (likely with antibiotics) via the selection marker integrated in the viral section of the transfer plasmid (gRNA or dCas9 plasmid). The selection marker, sgRNA/dCas9, gRNA target sequence/dCas9 activation domain, and viral segment of the plasmids (FIG. 3, panels A and B) are then integrated into the host genome as depicted in FIG. 3, panel E.

A lentiviral vector for expressing dCas9-VPR is commercially available (Dharmacon Edit-R CRISPa Lentiviral dCas9-VPR). This can be used together with another vector encoding the gRNA specific for the target gene, either as a single guide RNA molecule or as separate crRNA and tracrRNA molecules complexed together to form a guide RNA molecule.

Research has demonstrated that using glass as a substrate for fibroblast cells yields more collagen production than in culture. When cells are placed on a substrate that is not encountered in vivo, they produce collagen as a method of isolating themselves from the plate.

Fibroblasts also have a tendency to spread out collagen across a substrate of a higher stiffness, such as glass, rather than clumping. Following this, collagen layers would begin to stack under the cells on the plate. In most collagen producing studies, the amount of collagen produced is limited by the substrate size.

FIG. 11 shows a diagram of a glass plate array for fluid shear. The device can be utilized in a method to optimize the amount of surface area available for type I collagen growth and the amount of fluid shear applied across each surface. The number and cross-sectional area of glass slides can be determined by predictive models for CRISPR-based methods and vitamin/growth factor enhancements. The appropriate flow rate applied to the cells can be determined using fluid modeling software. A cost function could be used to draw a relationship between flow rate and magnitude of effect on collagen output.

The apparatus includes a series of stacked glass slides constrained on either fluid inlet/outlet end by jigs and watertight side walls on non-flow sides (FIG. 11). Using luer adapters and low-compliance tubing, fluid would be pushed across the plates using a pulsatile fluid pump.

Following flow shearing, the culture and collagen fibrils can be removed from the glass plates and the amount of type I collagen could be assayed to determine how much was produced.

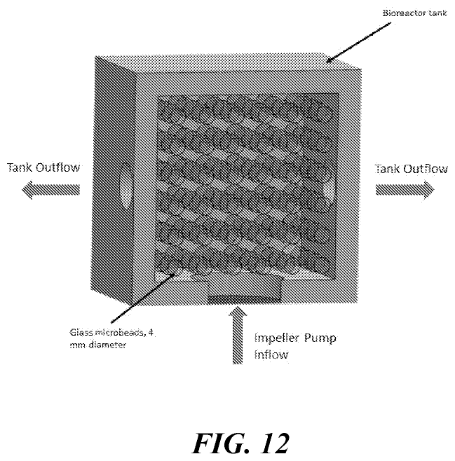

An alternative method of collagen growth using glass as a substrate is to utilize glass beads. An example device is depicted in FIG. 12. Microcarriers are advantageous in fibroblast growth applications because they provide maximal surface area for collagen growth while being able to pack into a full-immersion medium. Using an impellerto agitate the bead network and provide local flow to the fibroblasts, the system can provide shear stress to the growing network (FIG. 12). A proposed bioreactor can be compact and have one fluid inlet port and two fluid outlet ports. The flow rate supplied to the cells could be determined using fluid modeling software. Collagen would be extracted from the system following the removal of medium and measured to determine quantity.

### Examples

### Example 1. CRISPR Using Pooled Transfection

Three levels of transfection reagent were compared for a CRISPR design. The transfection reagent was DharmaFECT 1 Transfection Reagent as shown in Table 2. In all cell culture media, the media should be concentrated at least 10 times using a dialysis (50 Daltons molecular weight cutoff) against PBS to promote propeptide cleavage. The high, medium, and low, transfection reagent concentrations were 7.2, 4.05, and 0.9 μL of transfection reagent per well, respectively. Media was harvested every 12 hours and pooled in two-day segments. Collagen amount per well was quantified every two days, and cell counts were performed after the end of media harvesting. Collagen produced per cell was estimated based on the number of cells per well at the end of media collection. A comparison of total (days 1-6) pooled collagen production (per well, not per cell) for each concentration level and the cell count is shown in FIG. 5.

Quantifications that fell below the minimum detectable collagen amount were set to the minimum detectable collagen amount (1.06 million molecules/cell/hour). Having some quantifications fall below the minimum detectable amount was necessary to capture the upper limits of collagen production in the CRISPR conditions. In other words, we could have not diluted the samples before quantifying collagen, but then the upper limits of CRISPR collagen production would have been above the upper threshold for collagen quantification and not have been detectable. Note that days 1-2 data and most control samples were at or below the minimum detectable collagen amount, but every datapoint for CRISPR results for days 3-4 and 5-6 were above the minimum threshold. This is important for when fold change and statistical significance was calculated, as the actual collagen production for the control wells is likely much lower than what we are saying it is. Lastly, early timepoints (days 1-2) matching the control wells makes sense as the CRISPR was expected to upregulate collagen production after about two days. Based on the graph in FIG. 6, there are likely differences in collagen production both transiently and due to transfection reagent concentration.

An important feature of FIG. 7 is that there is no observed decrease in collagen production at the latest time point compared to earlier time points. Due to the fact that the CRISPR transfection method that was utilized results in a transient activation of collagen-related genes, an increase in collagen production is expected, compared to the control, followed by a decrease in collagen production. Here, only an increase in collagen production with time is observed, indicating that the peak timepoints for collagen production might not be observed. Thus, if later time points are tested, there very well could be an even greater increase in collagen production per cell.

The peak of collagen production seen using CRISPR showed about a 90.29-fold increase (42.1% std) in collagen production compared to the experimental control.

Cells that have been genetically modified with CRISPR can be cultured in media containing the top performing chemical conditions found in Example 2, including a control with CRISPR cells in traditional media and a second control.

In addition to the scale up methods depicted in FIG. 1, FIG. 2, and FIG. 3, a cost analysis of reagents needed for pooled transduction from Dharmacon was prepared in Table 20, and a CRISPR scale-up cost analysis with lentiviruses was prepared in Table 21. In Table 20, only one set of 4 lentiviral sgRNA purchase is necessary as target sgRNAs can be mixed and matched in one set of 4 purchase. Also, all crRNAs from Dharmacon cost the same amount, regardless of target gene. In Table 21, while the recurring cost of CRISPR reagents and transduction is effectively null, there are major costs associated with attaining, transducing, and validating collagen overexpression in a CRISPRed cell line.

TABLE 20
Reagents Needed for a Pooled Transduction.

| | Catalog Number |
|---|---|
| Item Name | (Dharmacon) |
| CRISPRa Human COL1A1 | GSGH11890- |
| Set of 4 | EG1277 [71] |
| Lentiviral sgRNA | |
| CRISPRa Human COL1A2 | GSGH11890- |
| Set of 4 | EG1278 [250] |
| Lentiviral sgRNA | |
| CRISPRa Human TGF-β3 | GSGH11890- |
| Set of 4 Lentiviral sgRNA | EG7040 [153] |
| QIAprep Spin Miniprep | U-002005-05-000 |
| Kit | |
| Trans-Lentiviral shRNA | TLP5912 [155] |
| Packaging Kit with | |
| Calcium Phosphate | |
| CRISPRa Lentiviral | T-2001-01 [251] |
| BlastdCas9-VPR Particles | |
| 10 mM Tris-HCl Buffer pH | B-006000-100 |
| 7.4 | |

### Example 2. Influence of Additives on Collagen Production

A variety of chemical stimulants were studied to be potentially used to increase the collagen production of fibroblast cells. Acetaldehyde, also known as ethanal, is a derivative of ethanol that has been shown to increase the levels of collagen produced in baboon liver myofibroblasts and human dermal fibroblasts when added to the culture media in concentrations up to 300 μM.

Ascorbic acid is known to be beneficial to the production of collagen in cell types including bovine, mouse, and human. It was hypothesized that the addition of caffeine to media could be beneficial to any lentiviral based CRISPR design solutions because of its demonstrated effect on increasing the activity of lentivirus in the gene therapy space. However, caffeine was shown to have a negative effect on collagen production in concentrations in media as little as 1-5 mM. This is most likely due to the inhibition of the accumulation of several growth factors, including interleukin-8.

While in high concentrations the addition of ethanol to cell culture media is commonly known to be extremely negative, a number of studies have been performed at concentrations of 50 mM in order to hypothesize the cause of alcoholic liver fibrosis. While fibrosis is commonly synonymous with an increase in the amount of collagen present in tissue, studies performed at these concentrations in myofibroblasts and fibroblasts report no direct effect on the level of collagen production.

Glutamate has not been studied extensively for its impact on collagen production despite its close relationship to glutamine, which has been known to be a very positive influence on collagen transcription rates. One study showed a 400% increase from the control in glutamate-supplemented media used to culture human dermal fibroblasts.

Many studies have been conducted in order to observe the response of human dermal fibroblasts in the presence of glutamine in media. Concentrations seen in these studies range up to 10 mM, demonstrating a maximum benefit for the production of collagen of nearly 300% of the control at 250 μM. This effect has been thought to increase the level of collagen gene transcription through its conversion into pyrroline-5-carboxylate.

Hyaluronic acid showed a neutral effect on collagen production in human dermal fibroblasts plated on an undisclosed surface at 500 pg/ml in media. It increased the rate of cell division and general fibril production when present at 150 pg/ml in media for human dermal fibroblast cultures plated on collagen, however researchers did not specifically quantify the production of collagen. When it was incorporated into the culture surface at a ratio of 1:19 hyaluronic acid to collagen by weight, it increased the amount of collagen produced by embryonic chick fibroblasts up to 230% of control. Based on the information presented in these studies and the well-known tendency of cells to stop producing collagen once enveloped in it, the group hypothesized that it interferes with a feedback process, fooling cells into making more collagen than they actually require for a suitable microenvironment.

There has been a demonstrated positive correlation between the level of Insulin-like Growth Factor 1 (IGF-1) in media and the production of collagen by manipulation of rat serum applied to media used to culture human dermal fibroblasts. Other studies quantify these values in human lung fibroblasts at a maximum of a 300% increase from control at a concentration of 100 ng/mL. Macroscopically, this effect can be seen in diabeteic individuals who are slow to heal wounds or suffer from accelerated atherosclerosis.

Interleukins (IL) encompass a wide range of glycoproteins associated with immune response. Researchers have looked into types 1p, 4, 6, 8, 10, and 13 for their specific effect on collagen production. IL-4 has demonstrated a maximum positive effect of a 250% increase from control. The lowest concentration of any of these types that is needed for an observable, positive effect is IL-1p at 2.5 μM in human chondrocytes. Out of the six types listed above, only IL-10 was found to reduce the level of collagen production.

Lactate is commonly found in high concentrations in the body after alcohol consumption, especially in individuals suffering from alcoholic liver fibrosis. Therefore, its addition into media could lead to an increase in collagen production even outside of the whole organ system. One study seems to agree with this logical argument by demonstrating a statistically significant increase in collagen production upon addition of 5 mM to media used to culture baboon liver myofibroblasts. However, in human dermal fibroblasts a concentration of 40 mM was shown to decrease collagen production.

Lathyrogens have been used to inhibit the formation of collagen crosslinks without cytotoxic effects. The most popular lathrogen used in cell culture is β-aminopropionitrile (BAPN) which operates by irreversibly blocking lysyl oxidase. Other cellular effects include prevented development of adhesive strength and a buildup of GuHCI-extractable collagen crosslink precursors. No research with any cell type has shown adverse effects on cell viability, collagen synthesis, or non collagen protein synthesis. However, one research study demonstrated inhibited fibroblast migration in a dose-dependent fashion at 0.25 and 0.5 mM BAPN. Previous research has used BAPN successfully at concentrations of 0.1 mM-0.5 mM.

Proline stabilizes collagen during post translational modifications. One study found a range of concentrations from 5-10 mM in media applied to human dermal fibroblasts that resulted in a maximum increase of 200% in collagen production as compared to the control value.

The effects of pyrroline-5-carboxylate on collagen production have been documented multiple times in human dermal fibroblasts. Information presented in these studies suggests an optimal concentration of 1 mM for a maximum increase of 260% of the control value. Interestingly, this effect can be seen in as little as 6 hours. It is thought to have such a potent effect because it enables IGF-1. Additionally, it can be converted to proline.

The subfamilies within the TGF-β family all have been shown to have a positive impact on collagen production. Generally, it has a positive effect on the number of ribosomes in the cell, the organelle responsible for the translation of all proteins, including collagen. TGF-β1 applied to rat liver M cells at a concentration in media as low as 1 ng/ml demonstrated an unquantified increase in collagen production from control. It has been shown that types of collagen produced by TGF-β vary, with collagen type I being especially associated with TGF-β3. Sources seem to agree on a concentration of 12.5 ng/ml for maximum efficacy in human dermal fibroblasts.

Based on a weighted scoring (Table 13), the seven best additives were selected and were added to the fibroblast media separately in a Phase 1 screening study using concentrations presented in Table 14. Table 14 shows the additives and concentrations to be tested. A common concentration cited in literature should be a standard condition, plus one concentration at 50% of that value, and another concentration at 150% that value (Table 14). Positive effects of these additives have been shown in other cell lines, but its main effects on corneal fibroblasts need to be explored independently before combinations of additives can be tested. This screening of factors phase is common in designed experiments in bioengineering applications. Specifically when using a factorial design for media composition it is recommended to perform an screening experiment of the unknown domain before applying advanced designs that allow optimization. Screening studies can be done in a number of ways. The simplest screening experimental design is each variable at two levels, however this assumes a linear relationship between the input and output. For this study a three-level design was chosen in order to determine a maxima. This phase of the experiment determined which additives have a statistically significant effect on collagen production and what concentration of each additive has the highest positive impact. This data was fed into Phase 2 of the experiment where the top performing additives were used within their optimal range of concentrations.

The seven additives also allowed for minimizing the number of plates and maximizing the number of used wells. This resulted in 3 plates, each with a standard media control group. In phase 1 of testing 7 media additives were tested at 3 concentrations with a sample size of 4 for each condition. Additives and concentrations used were determined by the prior research described above. Collagen amount per well was quantified, but cell counts were not performed. Collagen produced per cell was estimated based on the number of cells expected per well. The results are shown in Table 15. Some groups fell below the SirCol standard curve range of 1-50 ug. These values were represented as 1.00 ug/100 uL and were assigned the lowest rating for efficacy in the resulting trade study.

Three different top performing additives were identified (BAP, ACE, and ASC). Based on single additive performance, with only three media additives resulting in higher collagen production than the control, the three different additives went on to the DOE. For the three additives that advanced the highest ranking of each additive was used as the DOE centerpoint. Due to a lack of available stock, ACE low was substituted for ACE medium. As a result of these modifications, the final selections from Phase 1 were BAP low, acetaldehyde low, and ascorbate low for the centerpoint and all three at medium concentration for the “high” point (100%).

In phase 2 the top performing media additives were fed into a full factorial DOE. The concentrations used were determined by a variety of factors including a trade study, remaining laboratory supplies, and finally the lower cost associated with a lower concentration in a near-tie situation. A near-tie situation was defined by the group as concentrations of the same chemical that scored within 1% total score. For BAP, the concentration in the centerpoint was determined to be the low concentration from phase 1 at 0.25 mM due to it scoring the highest in the trade study. For ascorbate, a near-tie situation was observed so the low concentration from phase 1 at 0.5 mM was chosen. For acetaldehyde, due to a stock issue the low concentration at 0.2 mM was chosen. For the 100% concentration limit needed to complete construction of the full-factorial DOE plan, each of these values was doubled, which corresponds to the medium conditions from phase 1.

As shown in FIG. 9, only three conditions had collagen outputs over the limit of detection: ACS:BAP, ACS:BAP:ACE and the center point (CPT or 000). The fold increase for these three conditions was 1.96, 7.23 and 6.68, respectively. For these conditions the standard deviation was large. This may be due to cell death, pH or crystallization in the later two conditions. Because of these large variances there was no statistical difference between the groups. A larger sample size would be required to detect this difference.

### REFERENCES

Wu, F., Nerlich, M., & Docheva, D. (2017). Tendon injuries: Basic science and new repair proposals. EFORT open reviews, 2(7), 332-342.; doi.org/10.1302/2058-5241.2.160075.
(nhs.uk/conditions/hand-tendon-repair/recovery/) Recovery Hand Tendon Repair. (2018, June 14). Retrieved Feb. 13, 2020, from nhs.uk/conditions/hand-tendon-repair/recovery/.
(orthocarolina.com/media/how-does-a-patch-repair-a-rotator-cuff-tear) Rotator Cuff Surgery. (2018, May 21). Retrieved Feb. 16, 2020, from orthocarolina.com/media/how-does-a-patch-repair-a-rotator-cuff-tear.
Vig K, Chaudhari A, Tripathi S, Dixit S, Sahu R, Pillai S, Dennis V.A., Singh S.R. Advances in Skin Regeneration Using Tissue Engineering 18, 789, 2017.
Yao, J. Q., Blanchard, C. R., & Bloor, S. (2005). Zimmer© Collagen Repair Patch for Rotator
Cuff Tendon Repair. Zimmer, Inc.

OKeefe, R. J., Chu, C. R., Jacobs, J. J., & Einhorn, T. A. (2013). Orthopaedic basic science: foundations of clinical practice (4th ed.). Rosemont, IL: American Academy of Orthopaedic Surgeons.
Packer, H. (2016, February 04), reprinted with permission from CRISPR guide RNA format affects genome editing outcomes; retrieved from (idtdna.com/pages/education/decoded/article/crispr-guide-rna-format-affects-genome-editing-).
Biolabs: (neb.com, 2019), reprinted with permission from New England Biolabs from neb.com.
Biolabs, 2019: Biolabs, N. E. CRISPR/Cas9 & Targeted Genome Editing: New Era in Molecular Biology. Retrieved from (neb.com/tools-and-resources/feature-articles/crispr-cas9-and-targeted-genome-editing-a-new-era-in-molecular-biology).
Lodish H, Berk A, Zipursky SL, et al., Molecular Cell Biology. 4th edition. New York: W. H. Freeman; 2000. Section 22.3, “Collagen: The Fibrous Proteins of the Matrix”; available from: (ncbi.nlm.nih.gov/books/NBK21582/).
Schleip, R. (2012). Fascia: the tensional network of the human body the science and clinical applications in manual and movement therapy. Edinburgh: Churchill Livingstone/Elsevier.
NIH, COL1A1 and COL1A2 genes—Genetics Home Reference—(2019, May 28). Retrieved from (ghr.nlm.nih.gov/gene/COLlAl) and retrieved from (ghr.nlm.nih.gov/gene/COLlA2).
Gelse, K., Poschl, E., & Aigner, T. (2003). Collagens-structure, function, and biosynthesis. Advanced Drug Delivery Reviews. Available from: (core.ac.uk/download/pdf/2756387.pdf).
Wu M, Crane JS. Biochemistry, Collagen Synthesis. [Updated 2019 Apr 21]. In: StatPearls [Internet]. Treasure Island (FL): StatPearls Publishing; 2019 Jan—. Available from: (ncbi.nlm.nih.gov/books/NBK507709/).
Stuart, J. M., Townes, A. S., & Kang, A. H. (1982). Nature and specificity of the immune response to collagen in type II collagen-induced arthritis in mice. The Journal of clinical investigation, 69(3), 673-683. doi:10.1172/jci110495.
Lynn, A. K., Yannas, I. V., & Bonfield, W. (2004). Antigenicity and immunogenicity of collagen. Journal of Biomedical Materials Research, 71B(2), 343-354. doi:10.1002/jbm.b.30096.
Tuteja, R. (2005). Type I signal peptidase: An overview. Archives of Biochemistry and Biophysics. https://doi.org/10.1016/j.abb.2005.07.013.
Bonfanti, L., Mironov, A. A., Martinez-Menarguez, J. A., Martella, O., Fusella, A., Baldassarre, M., . . . Luini, A. (1998). Procollagen traverses the Golgi stack without leaving the lumen of cisternae: Evidence for cisternal maturation. Cell. https://doi.org/10.1016/S0092-8674(00)81723-7.
Uchio, Y., Ochi, M., Matsusaki, M., Kurioka, H., & Katsube, K. (2000). Human chondrocyte proliferation and matrix synthesis cultured in Atelocollagen gel. Journal of Biomedical Materials Research, 50(2), 138-143. doi:10.1002/(sici)1097-4636(200005)50:2<138::aid-jbm7>3.0.co;2-k.
CRISPR Guide. (n.d.). Retrieved from (addgene.org/crispr/guide/).
CRISPR Guide Design Software. (n.d.). Retrieved from (benchling.com/crispr/)
COL1A1 crRNA Pool: horizondiscovery.com/en/crisprmod/crispra/products/crispra-crrna?nodeid=entrezgene-1277
COL1A2 crRNA Pool:
   horizondiscovery.com/en/crisprmod/crispra/products/crispra-crrna?nodeid=entrezgene-1278
TGF-β3 crRNA Pool:
   horizondiscovery.com/en/crisprmod/crispra/products/crispra-crrna?nodeid=entrezgene-7043
Borawski, J., et al., Optimization Procedure for small interfering RNA Transfection in a 384-well format. J. Bimolecular Screening. 12, 546-559 (2007).
Pellegrini, A. (2016, February 1). How to Design gRNAs to Target Your Favorite Gene [Web log post]. Retrieved from (blog.benchling.com/2016/02/01/how-to-design-grnas-to-target-your-favorite-gene/).
Wyatt, D., & Ramsden, D. (2015, April 16). CRISPR 101: Non-Homologous End Joining [Web log post]. Retrieved from (blog.addgene.org/crispr-101-non-homologous-end-joining).
Mohr, S. E., Hu, Y., Ewen-Campen, B., Housden, B. E., Viswanatha, R., & Perrimon, N. (2016). CRISPR guide RNA design for research applications. The FEBS journal, 283(17), 3232-3238. https://doi.org/10.1111/febs.13777.
Zhang, Feng; Wen, Yan; Guo, Xiong; CRISPR/Cas9 for genome editing: progress, implications and challenges, Human Molecular Genetics, Volume 23, Issue R1,15 Sep. 2014, Pages R40-R46, (doi.org/10.1093/hmg/ddul25).
COL1A1 gene, 2019 —Genetics Home Reference—NIH. (2019, May 28). Retrieved from (ghr.nlm.nih.gov/gene/COL1A1).
COL1A2 gene, 2019 —Genetics Home Reference—NIH. (2019, May 28). Retrieved from (ghr.nlm.nih.gov/gene/COL1A2).
Introduction to Transfection. no date (n.d.). Retrieved from (thermofisher.com/us/en/home/references/gibco-cell-culture-basics/transfection-basics/introduction-to-transfection.html).
Human COL1A1 gene (1277). (n.d.). Retrieved from (dharmacon.horizondiscovery.com/biology-overview-entrezgene-1277-col1a1/).
MeSH Browser: Transduction, Genetic. (n.d.). Retrieved from (meshb.nlm.nih.gov/record/ui?name=Transduction, Genetic).
Edit-R CRISPRa crRNA. (n.d.). Retrieved from (dharmacon.horizondiscovery.com/crispr-activation/crispra-guide-rna/edit-r-crispra-crrna/?term=&p [73]005D982AF75C&sourceld=entrezgene/1277).
Multiplex CRISPR/Cas9-based genome engineering from a single lentiviral vector. (n.d.). Retrieved from (addgene.org/browse/article/8975/).
Kabadi, Ami M.; David G. Ousterout, Isaac B. Hilton, Charles A. Gersbach, Multiplex CRISPR/Cas9-based genome engineering from a single lentiviral vector, Nucleic Acids Research, Volume 42, Issue 19,29 October [145] 2014, Page e147, (doi.org/10.1093/nar/gku749).
Inspired Cell Solutions, 2018: Dharmacon™ Edit-R™ CRISPRa transcriptional activation system with synthetic guide RNA. (2018). Cambridge, MA: Horizon [150] Discovery Group Company.
Electroporation, 2015: Electroporation of Dharmacon™ Edit-R™ dCas9-VPR mRNA and synthetic guide RNA for gene modulation.. (2015, October). Retrieved from (dharmacon.horizondiscovery.com/uploadedFiles/Resources/dcas9-vpr-mrna-electroporation-protocol).
Transfection Protocol. (2016, September). Retrieved from (dharmacon.horizondiscovery.com/uploadedFiles/Resources/dcas9-vpr-mrna-quick-protocol.pdf).
Human TGFβ1 gene (7040). (n.d.). Retrieved from (dharmacon.horizondiscovery.com/biology-overview-entrezgene-7040-tgfb1/).
Transcriptional Activation System, 2018: Dharmacon™ Edit-R™ transcriptional activation system with expressed sgRNA. (2018). Retrieved from dharmacon.horizondiscovery.com/uploadedFiles/Resources/edit-r-crispra-lentiviral-guide-rna-manu).
Trans-Lentiviral shRNA Packaging System, 2016: Trans-Lentiviral shRNA Packaging System. (n.d.). Retrieved from (dharmacon.horizondiscovery.com/viral-packaging/trans-Ientiviral-shrna-packaging-system/).
Mandenius, C.-F. and Brundin, A. (2008), Bioprocess optimization using design-of-experiments methodology. Biotechnol Progress, 24: 1191-1203. doi:10.1002/btpr.67.
Wang Y.-H,Yang B,Ren J,Dong M.-L,Liang D,Xu A.-L. Optimization of medium composition for the production of clavulanic acid by Streptomyces clavuligerus. Process Biochem. 2004; 40:1161-1166.
Jinek, M., et al. A Programmable Dual-RNA-Guided DNA Endonuclease in Adaptive Bacterial Immunity. Science. 337(6096), 816-821 (2012).
Tanenbaum, M. E., et al. A Protein Tagging System for Signal Amplification in Gene Expression and Fluorescence Imaging. Cell 159(3):635-646 (2014).

What is claimed is:

1. An engineered cell capable of enhanced collagen biosynthesis, wherein the cell has been engineered to perform CRISPR-based activation (CRISPRa) of a targeted gene related to collagen biosynthesis by the cell, wherein the cell expresses an endonuclease deficient Cas9 (dCas9) protein fused to a transcriptional activator protein (dCas9-activator) and a guide RNA (gRNA) specific for the targeted gene, wherein the engineered cell is capable of at least 10-fold, at least 20-fold, at least 30-fold, at least 40-fold, at least 50-fold, at least 60-fold, at least 70-fold, at least 80-fold, at least 90-fold, at least 100-fold, at least 200-fold, or at least 300-fold higher collagen biosynthesis compared to a non-engineered cell of the same type.

2. The engineered cell of claim 1, wherein the targeted gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

3. The engineered cell of claim 2, wherein the gRNA expressed by the cell and specific for said targeted gene comprises the nucleotide sequence of any of SEQ ID NOS:1-156.

4. The engineered cell claim 1, wherein the cell has been engineered to perform CRISPRa of one or more further targeted genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.

5. The engineered cell of claim 1 that expresses gRNAs specific for two or more of said targeted genes, and each of the two or more targeted genes is activated.

6. The engineered cell of claim 5, wherein one or more collagen genes and one or more TGF β genes are targeted;
wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3;
wherein the one or more TGF-β genes are selected from the group consisting of TGF-β1, TGF-β2, and TGF-β3; and
wherein the cell expresses gRNAs specific for said one or more collagen genes and said one or more TGF-β genes.

7. The engineered cell of claim 6, wherein the collagen genes are selected from COL1A1 and COL1A2 and the TGF-β genes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:1 3-16, and the TGF-β3 gRNA comprises the nucleotide sequence of any of SEQ ID NOS:9-12.

8. The engineered cell of claim 6, wherein one or more propeptidase genes are further targeted, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

9. The engineered cell of claim 8, wherein the propeptidase genes ADAMTS2 and BMP-1 are targeted, and wherein the ADAMTS2 gRNA comprises the nucleotide sequence of any of SEQ ID NOS:69-72 and the BMP-1 gRNA comprises the nucleotide sequence of any of SEQ ID NOS:153-156.

10. The engineered cell of claim 1, wherein said transcriptional activator is selected from the group consisting of VP64, β65, Rta, VPR (a combination of VP64, β65, and Rta), MS2, HSF1, SAM (a combination of MS2, β65, and HSF-1), and SunTag.

11. The engineered cell of claim 10, wherein the dCas9-activator is dCas9-VPR.

12. The engineered cell of claim 1, wherein the cell has been transfected to express said dCas9-activator and said gRNA or gRNAs.

13. The engineered cell of claim 1, wherein the cell has been transduced to express said dCas9-activator and said gRNA or gRNAs.

14. The engineered cell of claim 1, wherein the cell is derived from a cell type selected from the group consisting of fibroblasts, mesenchymal cells, myofibroblasts, osteoblasts, chondrocytes, and induced pluripotent stem cells.

15. The engineered cell of claim 14, wherein the cell is derived from a human corneal fibroblast.

16. The engineered cell of claim 1, wherein the cell is derived from a cell obtained from a mammalian subject in need of collagen administration.

17. A cell culture comprising the engineered cell of claim 1.

18. The cell culture of claim 17 that is immortalized.

19. A method for engineering a cell to provide enhanced collagen biosynthesis, the method comprising the steps of:
(a) providing the cell, a first nucleic acid molecule encoding a dCas9-activator, and a second nucleic acid molecule specific for a target gene related to collagen biosynthesis; and
(b) transfecting or transducing the cell with said first and second nucleic acid molecules;
whereby the cell becomes capable of expressing said dCas9-activator and said gRNA, and the target gene is activated.

20. The method of claim 19, wherein the target gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1

21. The method of claim 20, wherein the gRNA comprises the nucleotide sequence of any of SEQ ID NO:1 to SEQ ID NO:156.

22. The method of claim 19, wherein the cell is engineered to perform CRISPRa of one or more further targeted genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.

23. The method of claim 19, wherein the cell is transfected with two or more second nucleic acid molecules, each specific for a different target gene, whereby each of the target genes is activated.

24. The method of claim 23, wherein one or more collagen genes and one or more TGF β genes are targeted;
wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3;
wherein said TGF-β genes are selected from the group consisting of TGF-β1, TGF-β2, and TGF-β3; and
wherein the cell expresses gRNAs specific for said one or more collagen genes and said one or more TGF-β genes.

25. The method of claim 24, wherein the collagen genes are selected from COL1A1 and COL1A2 and the TGF-β genes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:13-16, and the TGF-β3 gRNA comprises the nucleotide sequence of any of SEQ ID NOS:9-12.

26. The method of claim 24, wherein one or more propeptidase genes are further targeted, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

27. The method of claim 26, wherein the propeptidase genes ADAMTS2 and BMP-1 are targeted, and wherein the ADAMTS2 gRNA comprises the nucleotide sequence of any of SEQ ID NOS:69-72 and the BMP-1 gRNA comprises the nucleotide sequence of any of SEQ ID NOS:153-156.

28. The method of claim 19, wherein the dCas9-activator is dCas9-VPR.

29. The method of claim 19, wherein the cell is derived from a cell type selected from the group consisting of fibroblasts, myoblasts, osteoblasts, chondrocytes, and induced pluripotent stem cells.

30. The method of claim 19, wherein the cell is derived from a human corneal fibroblast.

31. The method of claim 19, wherein the cell is transduced using a lentiviral vector in step (b).

32. The method of claim 19, wherein step (a) includes obtaining a sample from a mammalian subject in need of collagen administration, or from a different mammalian subject of the same species, and deriving the provided cell from the sample.

33. A kit for engineering a cell to enhance biosynthesis of collagen by the cell, the kit comprising:
(i) a first nucleic acid molecule encoding a dCas9-activator protein;
(ii) a second nucleic acid molecule comprising or encoding a crRNA specific for a target gene related to collagen biosynthesis; and
(iii) optionally one or more reagents for transfecting or transducing a cell with the first and second nucleic acid molecules.

34. The kit of claim 33, wherein the target gene is selected from the group consisting of COL1A1, COL1A2, TGF-β1, TGF-β2, TGF-β3, COL2A1, COL3A1, COL4A1, COL4A2, COL4A3, COL4A4, COL4A5, COL4A6, COL5A1, COL5A2, COL5A3, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

35. The kit of claim 34, wherein the crRNA comprises the nucleotide sequence of any of SEQ ID NOS:1-156.

36. The kit of claim 33, wherein two or more second nucleic acid molecules are provided, each comprising or encoding a crRNA specific for a different target gene.

37. The kit of claim 36, wherein the two or more second nucleic acid molecules comprise or encode crRNAs specific for one or more collagen genes and one or more TGF β genes;
wherein the one or more collagen genes are selected from the group consisting of COL1A1, COL1A2, COL2A1, COL3A1, COL4A1, COL4A2, COL4A3, COL4A4, COL4A5, COL4A6, COL5A1, COL5A2, and COL5A3; and
wherein said TGF-β genes are selected from the group consisting of TGF-β1, TGF-β2, and TGF-β3.

38. The kit of claim 37, wherein the collagen genes are selected from COL1A1 and COL1A2 and the TGF-β genes are selected from TGF-β1 and TGF-β3, wherein the COL1A1 gRNA comprises the nucleotide sequence of any of SEQ ID NOS:1-4 and the COL1A2 gRNA comprises the nucleotide sequence of any of SEQ ID NOS:5-8, and wherein the TGF-β1 gRNA comprises the nucleotide sequence of any of SEQ ID NOS:13-16, and the TGF-β3 gRNA comprises the nucleotide sequence of any of SEQ ID NOS:9-12.

39. The kit of claim 37, wherein the two or more second nucleic acids further comprise or encode crRNAs specific for one or more propeptidase genes, wherein the propeptidase genes are selected from the group consisting of ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, TLL1, TLL2, and BMP1.

40. The kit of claim 39, wherein the two or more second nucleic acid molecules comprise or encode crRNAs specific for propeptidase genes ADAMTS2 and BMP-1, and wherein the ADAMTS2 gRNA comprises the nucleotide sequence of any of SEQ ID NOS:69-72 and the BMP-1 gRNA comprises the nucleotide sequence of any of SEQ ID NOS:153-156.

41. The kit of claim 33, wherein the second nucleic acid molecules further comprise or encode crRNAs specific for one or more genes selected from the group consisting of prolyl-3-hydroxylase family genes, prolyl-4-hydroxylase family genes, lysyl hydroxylase family genes, GLT25D1, GLT25D2, Grp78, Grp94, protein disulfide isomerase (PDI) family genes, calreticulin, calnexin, CypB, PPlase family genes, cyclophilins, FK506 binding protein (FKBP) genes, cyclophilin B (CypB), HSP47, TANG01, SEC13, SEC31, and Sedlin.

42. The kit of claim 33, wherein the dCas9-activator is dCas9-VPR.

43. A medical device comprising the engineered cell of claim 1.

44. The medical device of claim 43 that is implantable in a subject's body.

45. A method of producing collagen, the method comprising the steps of:
(a) providing the cell culture of claim 17;
(b) growing the cell culture in a bioreactor under conditions in which collagen is biosynthesized by the cells of the cell culture; and
(c) harvesting and purifying collagen from the bioreactor.

46. The method of claim 45, wherein step (b) is performed in the presence of a modulator of collagen biosynthesis.

47. The method of claim 46, wherein the modulator is selected from the group consisting of acetaldehyde, ascorbate, hyaluronic acid, β-aminopropionitrile, transforming growth factor beta (TGF-β), insulin-like growth factor 1 (IGF-1), glutamine, and combinations thereof.

48. The method of claim 47, wherein the modulator is a combination of ascorbate and p-aminopropionitrile or a combination of ascorbate, acetaldehyde, and β-aminopropionitrile.

49. The method of claim 47, wherein the modulator is β-aminopropionitrile, and wherein crosslinking of collagen is reduced or prevented compared to absence of β-aminopropionitrile.

50. The method of claim 45, wherein step (b) is performed in the presence of application of mechanical strain to the cells.

51. The method of claim 50, wherein mechanical strain is induced using cells adhered to a substrate, beads, or a scaffold.

52. The method of claim 45, further comprising, between steps (b) and (c):
(b1) concentrating the biosynthesized collagen in the cell growth medium, whereby propeptide cleavage of the biosynthesized collagen is enhanced.

53. The method of claim 45, wherein the collagen produced is a type selected from the group consisting of collagen types I-V.

54. The method of claim 53, wherein the collagen is type I collagen.

55. A method of treating a mammalian subject having a medical condition characterized by insufficiency of collagen, the method comprising:
(a) performing the method of claim 32 and thereby obtaining collagen produced by cells derived from the mammalian subject, or a different mammalian subject of the same species; and
(b) administering the collagen to the subject.

56. The method of claim 55, wherein a medical device is used to administer the collagen.

57. The method of claim 55, wherein the medical device is selected from the group consisting of a burn/wound covering or dressing, an osteogenic and/or bone filling material, a device having an antithrombogenic surface, a device having a therapeutic enzyme immobilization surface, a collagen patch, a closure graft, an implant operative to provide collagen, a corneal implant, a bandage contact lens, a collagen-based membrane, and a collagen-based drug delivery device.

58. The method of claim 55, wherein the medical condition is selected from the group consisting of a wound, a torn ligament or tendon, a bone fracture, damaged cartilage, an eye condition, a condition requiring cosmetic treatment or surgery, a dermatological condition, skin wrinkles or scars, and a burn.

59. A method of performing a cosmetic treatment to a human subject, the method comprising:
(a) performing the method of claim 32, thereby obtaining collagen produced by cells derived from the mammalian subject or other subject of the same mammalian species; and
(b) administering the collagen obtained in step (a) to the subject.


SEQUENCE LISTING (162 sequences)

SEQ ID NO: 1 - DNA, 20 bp, Homo sapiens
gctgcgaagaggggagatgt

SEQ ID NO: 2 - DNA, 20 bp, Homo sapiens
ggggaggcagagctgcgaag

SEQ ID NO: 3 - DNA, 20 bp, Homo sapiens
ccggcccccaatttgggagt

SEQ ID NO: 4 - DNA, 20 bp, Homo sapiens
ggaaccctgcccctcggaga

SEQ ID NO: 5 - DNA, 20 bp, Homo sapiens
aagggcctccaccaatggga

SEQ ID NO: 6 - DNA, 20 bp, Homo sapiens
cgcagaggagggagcgaatg

SEQ ID NO: 7 - DNA, 20 bp, Homo sapiens
ggggaagggacgtggccacg

SEQ ID NO: 8 - DNA, 20 bp, Homo sapiens
gggagggcgggaggatgcgg

SEQ ID NO: 9 - DNA, 20 bp, Homo sapiens
ttaacatcgtgcagcaaaag

SEQ ID NO: 10 - DNA, 20 bp, Homo sapiens
gagggcgcgggacccggtag

SEQ ID NO: 11 - DNA, 20 bp, Homo sapiens
gcaaaagaggctgcgtgcgc

SEQ ID NO: 12 - DNA, 20 bp, Homo sapiens
ccgggaccgggggaccagga

SEQ ID NO: 13 - DNA, 20 bp, Homo sapiens
gcggccaagcgccaccaaag

SEQ ID NO: 14 - DNA, 20 bp, Homo sapiens
gagcccgcccacgcgagatg

SEQ ID NO: 15 - DNA, 20 bp, Homo sapiens
ccccgcgggcggctcagagc

SEQ ID NO: 16 - DNA, 20 bp, Homo sapiens
ccgcccacgcgagatgagga

SEQ ID NO: 17 - DNA, 20 bp, Homo sapiens
gagctctccccgaaccgttg

SEQ ID NO: 18 - DNA, 20 bp, Homo sapiens
atgaggaccgctgtgggtaa

SEQ ID NO: 19 - DNA, 20 bp, Homo sapiens
gtggaaatgaggaccgctgt

SEQ ID NO: 20 - DNA, 20 bp, Homo sapiens
ctcgtggtctaagtaacgag

SEQ ID NO: 21 - DNA, 20 bp, Homo sapiens
gccgcagccccgggtttggg

SEQ ID NO: 22 - DNA, 20 bp, Homo sapiens
gccactcggcgcactagggg

SEQ ID NO: 23 - DNA, 20 bp, Homo sapiens
caggccactcggcgcactag

SEQ ID NO: 24 - DNA, 20 bp, Homo sapiens
ccaagccggacccccctctc

SEQ ID NO: 25 - DNA, 20 bp, Homo sapiens
cggctctcatatttcagaaa

SEQ ID NO: 26 - DNA, 20 bp, Homo sapiens
gcagttgttaacttcataag

SEQ ID NO: 27 - DNA, 20 bp, Homo sapiens
tgtgggttgtgtcttctata

SEQ ID NO: 28 - DNA, 20 bp, Homo sapiens
taacttctaggacccagggt

SEQ ID NO: 29 - DNA, 20 bp, Homo sapiens
gggcgcgaggggttgggacg

SEQ ID NO: 30 - DNA, 20 bp, Homo sapiens
gaccctgcggcgcgggtaag

SEQ ID NO: 31 - DNA, 20 bp, Homo sapiens
gagcgcgcggcccgggagtg

SEQ ID NO: 32 - DNA, 20 bp, Homo sapiens
gcgcactgcagccacactcc

SEQ ID NO: 33 - DNA, 20 bp, Homo sapiens
tggagccgccgcacccggga

SEQ ID NO: 34 - DNA, 20 bp, Homo sapiens
ccgggtgcggcggctccaag

SEQ ID NO: 35 - DNA, 20 bp, Homo sapiens
gcggacagctagctctcgga

SEQ ID NO: 36 - DNA, 20 bp, Homo sapiens
gccccatggtggcgcgcccg

SEQ ID NO: 37 - DNA, 20 bp, Homo sapiens
cgtgcccaggaggcgagaaa

SEQ ID NO: 38 - DNA, 20 bp, Homo sapiens
tctacccgggcatcgtgccc

SEQ ID NO: 39 - DNA, 20 bp, Homo sapiens
ggcacgatgcccgggtagaa

SEQ ID NO: 40 - DNA, 20 bp, Homo sapiens
agggacactgcctggtaagt

SEQ ID NO: 41 - DNA, 20 bp, Homo sapiens
cgtgcccaggaggcgagaaa

SEQ ID NO: 42 - DNA, 20 bp, Homo sapiens
tctacccgggcatcgtgccc

SEQ ID NO: 43 - DNA, 20 bp, Homo sapiens
gggaagtggggtgcggtcgg

SEQ ID NO: 44 - DNA, 20 bp, Homo sapiens
ggatccagggtaaggggtta

SEQ ID NO: 45 - DNA, 20 bp, Homo sapiens
gagtgacgctcagttatttg

SEQ ID NO: 46 - DNA, 20 bp, Homo sapiens
gactgcaccggcaacctgcg

SEQ ID NO: 47 - DNA, 20 bp, Homo sapiens
ggtacgcacaccaatgagat

SEQ ID NO: 48 - DNA, 20 bp, Homo sapiens
gtcactccctcgcaggttgc

SEQ ID NO: 49 - DNA, 20 bp, Homo sapiens
ttagcgtataggtctctaag

SEQ ID NO: 50 - DNA, 20 bp, Homo sapiens
cgggcccatctgtcttatgt

SEQ ID NO: 51 - DNA, 20 bp, Homo sapiens
ctaccggctgcccaaggtag

SEQ ID NO: 52 - DNA, 20 bp, Homo sapiens
ttgcttaggtcttagcgtat

SEQ ID NO: 53 - DNA, 20 bp, Homo sapiens
ggtacgcacaccaatgagat

SEQ ID NO: 54 - DNA, 20 bp, Homo sapiens
gagtgacgctcagttatttg

SEQ ID NO: 55 - DNA, 20 bp, Homo sapiens
gactgcaccggcaacctgcg

SEQ ID NO: 56 - DNA, 20 bp, Homo sapiens
gccggtgcagtctaaaactg

SEQ ID NO: 57 - DNA, 20 bp, Homo sapiens
cgggcgagtcgcagcgagga

SEQ ID NO: 58 - DNA, 20 bp, Homo sapiens
cccggggcggagcggacgtg

SEQ ID NO: 59 - DNA, 20 bp, Homo sapiens
cctcgcccgcggcgcccagt

SEQ ID NO: 60 - DNA, 20 bp, Homo sapiens
ccccaggcccgcccgcctac

SEQ ID NO: 61 - DNA, 20 bp, Homo sapiens
ccaagcaacggtctgattga

SEQ ID NO: 62 - DNA, 20 bp, Homo sapiens
cagagacgcgtgttctgatt

SEQ ID NO: 63 - DNA, 20 bp, Homo sapiens
aaagttaaagggtgtgtgtc

SEQ ID NO: 64 - DNA, 20 bp, Homo sapiens
ttaacttttaagcatagatg

SEQ ID NO: 65 - DNA, 20 bp, Homo sapiens
actggaaccctcgaactcta

SEQ ID NO: 66 - DNA, 20 bp, Homo sapiens
tgcacctcctcctaattcta

SEQ ID NO: 67 - DNA, 20 bp, Homo sapiens
ggaggctgaagtcttgaatg

SEQ ID NO: 68 - DNA, 20 bp, Homo sapiens
aacaacccagctcctggcgc

SEQ ID NO: 69 - DNA, 20 bp, Homo sapiens
gcggccgcgccaagatgcgc

SEQ ID NO: 70 - DNA, 20 bp, Homo sapiens
gcgaagagggaagcgggcgg

SEQ ID NO: 71 - DNA, 20 bp, Homo sapiens
cgagcccggcaccgcggcga

SEQ ID NO: 72 - DNA, 20 bp, Homo sapiens
gcagagacaccccgaggcgg

SEQ ID NO: 73 - DNA, 20 bp, Homo sapiens
cgacccgcggggcgctaata

SEQ ID NO: 74 - DNA, 20 bp, Homo sapiens
ctccgcctagggcgagagga

SEQ ID NO: 75 - DNA, 20 bp, Homo sapiens
cgcgccccccgcaccgtgtc

SEQ ID NO: 76 - DNA, 20 bp, Homo sapiens
gttcgggccccgcatgacgt

SEQ ID NO: 77 - DNA, 20 bp, Homo sapiens
ggggctctgtcccaccaaaa

SEQ ID NO: 78 - DNA, 20 bp, Homo sapiens
aaaaaatgggacttgcccag

SEQ ID NO: 79 - DNA, 20 bp, Homo sapiens
acagctgagggctgattgtg

SEQ ID NO: 80 - DNA, 20 bp, Homo sapiens
ctagcagccgaatggataat

SEQ ID NO: 81 - DNA, 20 bp, Homo sapiens
gtccgcgcctaatcagatgg

SEQ ID NO: 82 - DNA, 20 bp, Homo sapiens
gaggagggtgatcgaggaaa

SEQ ID NO: 83 - DNA, 20 bp, Homo sapiens
gcaaaagaggagggtgatcg

SEQ ID NO: 84 - DNA, 20 bp, Homo sapiens
aggcgcggactgggaagggt

SEQ ID NO: 85 - DNA, 20 bp, Homo sapiens
gggaggtagaggtacaatcg

SEQ ID NO: 86 - DNA, 20 bp, Homo sapiens
agatgtgcctgggctgcgtc

SEQ ID NO: 87 - DNA, 20 bp, Homo sapiens
ccccctccacgtgacgaccc

SEQ ID NO: 88 - DNA, 20 bp, Homo sapiens
gtcctgggtcgtcacgtgga

SEQ ID NO: 89 - DNA, 20 bp, Homo sapiens
gcaggccgtgctcgcctcaa

SEQ ID NO: 90 - DNA, 20 bp, Homo sapiens
gcgcaggcagcgccccgcaa

SEQ ID NO: 91 - DNA, 20 bp, Homo sapiens
cacggcctgcggggccgatg

SEQ ID NO: 92 - DNA, 20 bp, Homo sapiens
aggggggcggacatccgttg

SEQ ID NO: 93 - DNA, 20 bp, Homo sapiens
caccccgggaagcaccgagt

SEQ ID NO: 94 - DNA, 20 bp, Homo sapiens
gatcccaggggaggggaaac

SEQ ID NO: 95 - DNA, 20 bp, Homo sapiens
gaaagcggttggggtctccc

SEQ ID NO: 96 - DNA, 20 bp, Homo sapiens
tcctctgcggccaagagtcc

SEQ ID NO: 97 - DNA, 20 bp, Homo sapiens
gaggaaaaagagactcggaa

SEQ ID NO: 98 - DNA, 20 bp, Homo sapiens
gccgaggaggggacatggtc

SEQ ID NO: 99 - DNA, 20 bp, Homo sapiens
ccccggtgcacgcctctaag

SEQ ID NO: 100 - DNA, 20 bp, Homo sapiens
gaggccctgccggctgcaag

SEQ ID NO: 101 - DNA, 20 bp, Homo sapiens
ggcggggcggcccgagttcc

SEQ ID NO: 102 - DNA, 20 bp, Homo sapiens
gcgcccccgccgcgtgggag

SEQ ID NO: 103 - DNA, 20 bp, Homo sapiens
agccagggacccgggaactc

SEQ ID NO: 104 - DNA, 20 bp, Homo sapiens
gggcggggaggggacgaagc

SEQ ID NO: 105 - DNA, 20 bp, Homo sapiens
gaggccgcggggcatgcggg

SEQ ID NO: 106 - DNA, 20 bp, Homo sapiens
gacagtgtccgctttcggcg

SEQ ID NO: 107 - DNA, 20 bp, Homo sapiens
ctggccgagccggccaaata

SEQ ID NO: 108 - DNA, 20 bp, Homo sapiens
ccggccaaataggggagacc

SEQ ID NO: 109 - DNA, 20 bp, Homo sapiens
gtgcacgcccacccccttag

SEQ ID NO: 110 - DNA, 20 bp, Homo sapiens
tctaagatggttgcagttca

SEQ ID NO: 111 - DNA, 20 bp, Homo sapiens
gggagcggggaccccggaga

SEQ ID NO: 112 - DNA, 20 bp, Homo sapiens
gcaggggccgtggctagggt

SEQ ID NO: 113 - DNA, 20 bp, Homo sapiens
caggccctgcggccaagaaa

SEQ ID NO: 114 - DNA, 20 bp, Homo sapiens
ggggatcccaggcgtctgag

SEQ ID NO: 115 - DNA, 20 bp, Homo sapiens
ccgcccgcgcagtgacgctc

SEQ ID NO: 116 - DNA, 20 bp, Homo sapiens
ggctttgggccaccactccg

SEQ ID NO: 117 - DNA, 20 bp, Homo sapiens
gtggctctccgctctggagg

SEQ ID NO: 118 - DNA, 20 bp, Homo sapiens
ggagagccacttggcgggga

SEQ ID NO: 119 - DNA, 20 bp, Homo sapiens
gccactccctccccgccaag

SEQ ID NO: 120 - DNA, 20 bp, Homo sapiens
cagagcggagagccacttgg

SEQ ID NO: 121 - DNA, 20 bp, Homo sapiens
gcccctgtggtcaacctcgt

SEQ ID NO: 122 - DNA, 20 bp, Homo sapiens
tcggaattccgagaagaatc

SEQ ID NO: 123 - DNA, 20 bp, Homo sapiens
ctgcctacgaggttgaccac

SEQ ID NO: 124 - DNA, 20 bp, Homo sapiens
gggacacgtaatcgcccctg

SEQ ID NO: 125 - DNA, 20 bp, Homo sapiens
gggaggaggcgaggtcagcg

SEQ ID NO: 126 - DNA, 20 bp, Homo sapiens
aggtcagcggggcgctgagg

SEQ ID NO: 127 - DNA, 20 bp, Homo sapiens
gaccgaggaggggagagtgc

SEQ ID NO: 128 - DNA, 20 bp, Homo sapiens
ccgcgcggggaccacacagt

SEQ ID NO: 129 - DNA, 20 bp, Homo sapiens
ggcggggcggaggaaagcga

SEQ ID NO: 130 - DNA, 20 bp, Homo sapiens
tcgacccatcccaaccagta

SEQ ID NO: 131 - DNA, 20 bp, Homo sapiens
gacaacctaactacgcactg

SEQ ID NO: 132 - DNA, 20 bp, Homo sapiens
ttcccctcggaaggcgggag

SEQ ID NO: 133 - DNA, 20 bp, Homo sapiens
gaggctgggtgggagtgata

SEQ ID NO: 134 - DNA, 20 bp, Homo sapiens
agggaccgaccgccatgaga

SEQ ID NO: 135 - DNA, 20 bp, Homo sapiens
ccgggctggtgcaagggcgt

SEQ ID NO: 136 - DNA, 20 bp, Homo sapiens
gtgcaagggcgtgggattcc

SEQ ID NO: 137 - DNA, 20 bp, Homo sapiens
taattcgccagggagctcga

SEQ ID NO: 138 - DNA, 20 bp, Homo sapiens
gagggaatgagtagggagat

SEQ ID NO: 139 - DNA, 20 bp, Homo sapiens
ggctgtgggtctgtcttgtg

SEQ ID NO: 140 - DNA, 20 bp, Homo sapiens
ggcgcgggagggaatgagta

SEQ ID NO: 141 - DNA, 20 bp, Homo sapiens
agggtaccgcagtccccttg

SEQ ID NO: 142 - DNA, 20 bp, Homo sapiens
tgcggctgagtggagaaggg

SEQ ID NO: 143 - DNA, 20 bp, Homo sapiens
ttggccgggaacagcccata

SEQ ID NO: 144 - DNA, 20 bp, Homo sapiens
gcgcggggaaaagcgagcag

SEQ ID NO: 145 - DNA, 20 bp, Homo sapiens
gtaagcaggatgcaagtgat

SEQ ID NO: 146 - DNA, 20 bp, Homo sapiens
ttggctaggggcccacgggt

SEQ ID NO: 147 - DNA, 20 bp, Homo sapiens
gagcaaaagtgtgaggattt

SEQ ID NO: 148 - DNA, 20 bp, Homo sapiens
ggggagggcgcaggcaaact

SEQ ID NO: 149 - DNA, 20 bp, Homo sapiens
cggcaccttcgcaacttcgg

SEQ ID NO: 150 - DNA, 20 bp, Homo sapiens
gcctcgggcgcccgagtgat

SEQ ID NO: 151 - DNA, 20 bp, Homo sapiens
tgctcctgaagatcgggact

SEQ ID NO: 152 - DNA, 20 bp, Homo sapiens
gcgccccgggcaggcgcctt

SEQ ID NO: 153 - DNA, 20 bp, Homo sapiens
cccgccccccgctcggtccg

SEQ ID NO: 154 - DNA, 20 bp, Homo sapiens
cggagccgcggaccgagcgg

SEQ ID NO: 155 - DNA, 20 bp, Homo sapiens
ggcccggccgagcactgtcc

SEQ ID NO: 156 - DNA, 20 bp, Homo sapiens
cactgtcccggccccgaggg

SEQ ID NO: 157 - protein, 1368 aa, Staphylococcus aureus
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 158 - RNA, 86 nt, Artificial Sequence
  Trans-activating crRNA (tracrRNA)
ggaaccauucaaaacagcauagcaaguuaaaauaaggcuaguccguuaucaacuugaaaaaguggcaccgagucggugcuuuuuuu

SEQ ID NO: 159 - DNA, 150 bp, Artificial sequence
  VP64 transcriptional activator coding sequence
gacgctttggacgacttcgacttggacatgttgggttctgacgctttggacgacttcgacttggacatgttgggttctgacgctttggacgacttcgacttggacatgttgggttctgacgctttggacgacttcgacttggacatgttg

SEQ ID NO: 160 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 161 - protein, 529 aa, Homo sapiens
MDLPVGPGAAGPSNVPAFLTKLWTLVSDPDTDALICWSPSGNSFHVFDQGQFAKEVLPKYFKHNNMASFVRQLNMYGFRKVVHIEQGGLVKPERDDTEFQHPCFLRGQEQLLENIKRKVTSVSTLKSEDIKIRQDSVTKLLTDVQLMKGKQECMDSKLLAMKHENEALWREVASLRQKHAQQQKVVNKLIQFLISLVQSNRILGVKRKIPLMLNDSGSAHSMPKYSRQFSLEHVHGSGPYSAPSPAYSSSSLYAPDAVASSGPIISDITELAPASPMASPGGSIDERPLSSSPLVRVKEEPPSPPQSPRVEEASPGRPSSVDTLLSPTALIDSILRESEPAPASVTALTDARGHTDTEGRPPSPPPTSTPEKCLSVACLDKNELSDHLDAMDSNLDNLQTMLSSHGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 162 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY